(19) United States
(12) Patent Application Publication
(10) Pub. No.: US 2024/0043456 A1
(43) Pub. Date: Feb. 8, 2024

(54) Crystalline Solid Meglumine Salt Inhibitor of BCL and Methods of Making and Using Same

(71) Applicant: Unity Biotechnology, Inc., South San Francisco, CA (US)

(72) Inventors: Liang Liao, South San Francisco, CA (US); Yuxi Ning, Beijing (CN); Jane Li, Beijing (CN); Yue Lu, Beijing (CN); Albert Shi, South San Francisco, CA (US); Nathan Guz, South San Francisco, CA (US)

(21) Appl. No.: 18/032,944

(22) PCT Filed: Nov. 10, 2020
(86) PCT No.: PCT/CN2020/127666

§ 371 Date: Apr. 20, 2023

(51) Int. Cl. C07F 9/6558

(52) U.S. Cl. CPC C07F 9/65583; C07B 2200/13

(57) ABSTRACT

Provided are the crystalline solid meglumine salts of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3- ((trifluoromethyl)sulfonyl)phenyl)sulfonamido)phenyl) piperazin-1-yl)phenyl)-1H-pyrrole-3-carboxylic acid. Pharmaceutical compositions having one or more of the crystalline solid meglumine salt compounds and methods for administering the crystalline solid meglumine salt compounds to a subject are also described. Methods for preparing the crystalline solid meglumine salt compounds are also provided.

### BACKGROUND

Normally mitotic cells can permanently withdraw from the cell cycle in response to cellular stress, including dysfunctional telomeres, DNA damage, strong mitogenic signals, and disrupted chromatin. This response is termed cellular senescence and has been shown to be important to inhibiting proliferation of dysfunctional or damaged cells and particularly to constraining development of cancer malignancy mechanisms (see Campisi J., Cell 120:513-22 (2005); Campisi J., Curr. Opin. Genet. Dev. 21: 107-12 (2011)). Senescent cells are characterized by numerous cellular phenotypes, including insensitivity to mitogenic stimuli, flattened morphology, increased senescence-associated β-galactosidase activity (SA-β-gal), elevated p16 expression, shortened telomeres, elevated cyclin-dependent kinase inhibitor expression, changes in chromatin structure, pervasive DNA damage foci, resistance to apoptosis and activation of the pro-inflammatory senescence-associated secretory phenotype (SASP) (see Coppe, J-P, et al., Annu Rev Pathol. 2010; 5: 99-118).

Recently, the presence and accumulation of senescent cells in an individual may contribute to aging and aging-related dysfunction and diseases, such as, for example, glaucoma, cataracts, diabetic pancreas and osteoarthritis, among others (see van Deursen J M., Nature. 2014 May 22; 509 (7501): 439-446; Childs, B. et al., Nat Med. 2015 December; 21(12): 1424-1435).

Given that senescent cells have been causally implicated in certain aspects of age-related decline in health and likely contributes to certain age-related diseases, including cancer, effective therapeutics are being researched and developed. Small-molecule drugs have been identified that selectively remove accumulated senescent cells in and around the affected area, alleviating adverse signs and symptoms of the resulting conditions. Several intracellular pathways that are active in senescent cells have been shown to be amenable to targeting, such as, for example, the MDM2 pathway, the Bel pathway, the Akt pathway, and the proteasome pathway, among others (see WO/2015/171591: Zhou et al.; WO/2015/116740: Laberge et al.; WO/2019/133904: Hudson et al.). The present disclosure addresses these needs and offers advantages.

### SUMMARY

Aspects of the disclosure include crystalline solid meglumine salts of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid. Pharmaceutical compositions having one or more of the subject crystalline solid meglumine salt compounds and methods for administering the crystalline solid meglumine salt compounds to a subject are also described. Methods for preparing the subject crystalline sold meglumine salt compounds are also provided.

Aspect 1. A crystalline solid meglumine salt of of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid, the compound of Formula I:

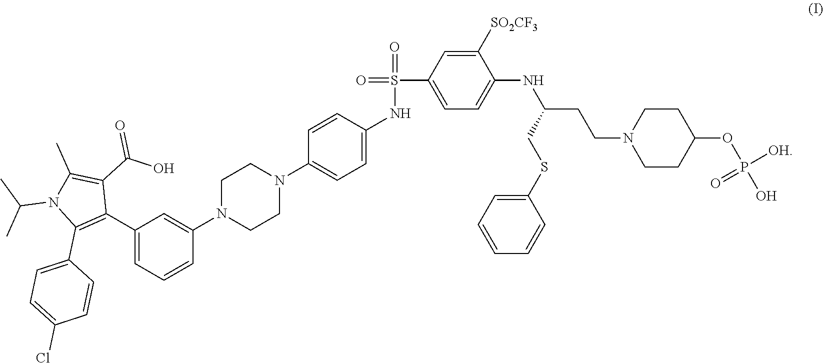

Aspect 2. The crystalline solid of aspect 1, wherein meglumine is present in the crystalline solid in a stoichiometric ratio of from 1 to 3.

Aspect 3. The crystalline solid of any one of aspects 1-2, wherein the crystalline solid is stable at a temperature of from 2° C. to 8° C. for 12 months or more.

Aspect 4. Form I of a crystalline solid meglumine salt of a compound of Formula I:

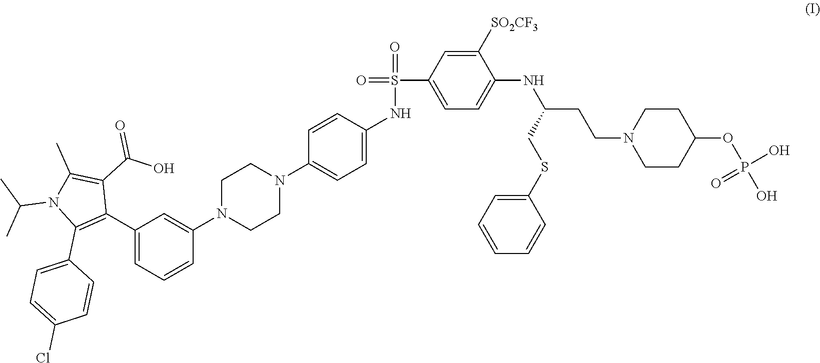

Aspect 5. The crystalline solid of aspect 4, wherein meglumine is present in the crystalline solid in a stoichiometric ratio of from 1 to 3.

Aspect 6. The crystalline solid of any one of aspects 4-5, having an x-ray powder diffraction pattern comprising one or more peaks at about 4.3° 2θ; about 6.1° 2θ; about 8.1° 2θ; about 8.6° 2θ; about 9.0° 2θ; about 10.1° 2θ; about 11.3° 2θ; about 12.2° 2θ; about 15.2° 2θ; about 16.2° 2θ; about 17.3° 2θ; about 18.2° 2θ; about 18.9° 2θ; about 19.3° 2θ; about 19.8° 2θ; about 20.7° 2θ; about 21.6° 2θ; about 22.1° 2θ; about 23.0° 2θ; about 24.2° 2θ; about 25.2° 2θ; about 25.5° 2θ; about 26.1° 2θ; about 27.1° 2θ; about 29.5° 2θ; or about 3.2.6° 20.

Aspect 7. The crystalline solid of any one of aspects 4-6, wherein Form 1 of the crystalline solid meglumine salt of a compound of Formula I is characterized by a 0.9% weight loss step of by thermogravimetric analysis (TGA) between room temperature to 130° C. and a second weight loss step at about 130° C.

Aspect 8. The crystalline solid of any one of aspects 4-7, wherein Form I of the crystalline solid meglumine salt of a compound of Formula I exhibits a first endotherm at 84° C. and a second endotherm at about 147° C. by differential scanning calorimetry (DSC).

Aspect 9. The crystalline solid of any one of aspects 4-8, wherein the crystalline solid is stable at a temperature of from 2° C. to 8° C. for 12 months or more.

Aspect 10. Form II of a crystalline solid meglumine salt of a compound of Formula I:

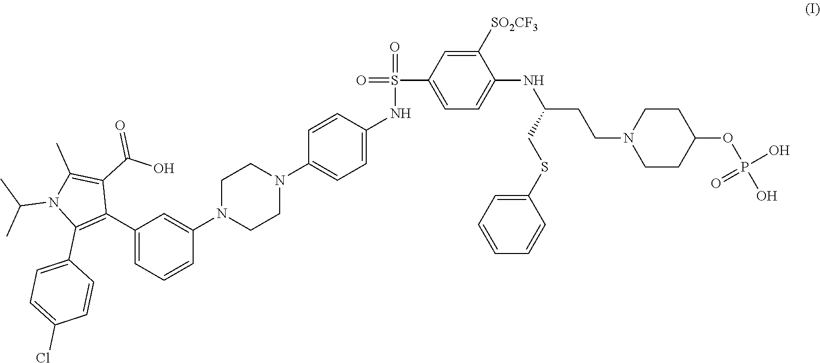

Aspect 11. The crystalline solid of aspect 10, wherein meglumine is present in the crystalline solid in a stoichiometric ratio of from 1 to 3.

Aspect 12. The crystalline solid of any one of aspects 10-11, having an x-ray powder diffraction pattern comprising one or more peaks at about 3.8° 2θ; about 7.3° 2θ; about 8.3° 2θ; about 8.8° 2θ; about 13.7° 2θ; about 15.2° 2θ; about 15.4° 2θ; about 16.6° 2θ; about 17.7° 2θ; about 18.8° 2θ; about 20.0° 2θ; about 22.1° 2θ; or about 23.9° 2θ.

Aspect 13. The crystalline solid of any one of aspects 10-12, wherein Form II of the crystalline solid meglumine salt of a compound of Formula I is characterized by a 2.0% weight loss step of by thermogravimetric analysis (TGA) between room temperature to 130° C. and a second weight loss step at about 130° C.

Aspect 14. The crystalline solid of any one of aspects 10-13, wherein Form II of the crystalline solid meglumine salt of a compound of Formula I exhibits an endotherm at about 136° C. by differential scanning calorimetry (DSC).

Aspect 15. The crystalline solid of any one of aspects 10-14, wherein the crystalline solid is stable at a temperature of from 2° C. to 8° C. for 12 months or more.

Aspect 16. Form III of a crystalline solid meglumine salt of a compound of Formula I:

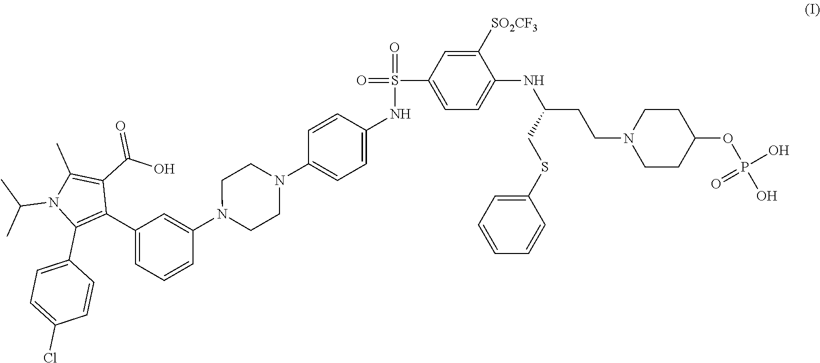

Aspect 17. The crystalline solid of aspect 16, wherein meglumine is present in the crystalline solid in a stoichiometric ratio of from 1 to 3.

Aspect 18. The crystalline solid of any one of aspects 16-17, having an x-ray powder diffraction pattern comprising one or more peaks at about 3.9° 2θ; about 4.3° 2θ; about 6.1° 2θ; about 7.5° 2θ; about 7.7° 2θ; about 8.7° 2θ; about 10.4° 2θ; about 11.3° 2θ; about 11.5° 2θ; about 12.5° 2θ; about 13.9° 2θ; about 14.7° 2θ; about 15.2° 2θ; about 15.9° 2θ; about 17.7° 2θ; about 18.0° 2θ; about 18.8° 2θ; about 20.2° 2θ; about 21.7° 2θ; about 23.0° 2θ; or about 25.8° 2θ.

Aspect 19. The crystalline solid of any one of aspects 16-18, wherein Form III of the crystalline solid meglumine salt of a compound of Formula I is characterized by a 0.9% weight loss step of by thermogravimetric analysis (TGA) between room temperature to 130° C. and a second weight loss step at about 130° C.

Aspect 20. The crystalline solid of any one of aspects 16-19, wherein Form III of the crystalline solid meglumine salt of a compound of Formula I exhibits a first endotherm at about 113° C. and a second endotherm at about 142° C. by differential scanning calorimetry (DSC).

Aspect 21. The crystalline solid of any one of aspects 16-20, wherein the crystalline solid is stable at a temperature of from 2° C. to 8° C. for 12 months or more.

Aspect 22. Form IVa of a crystalline solid meglumine salt of a compound of Formula I:

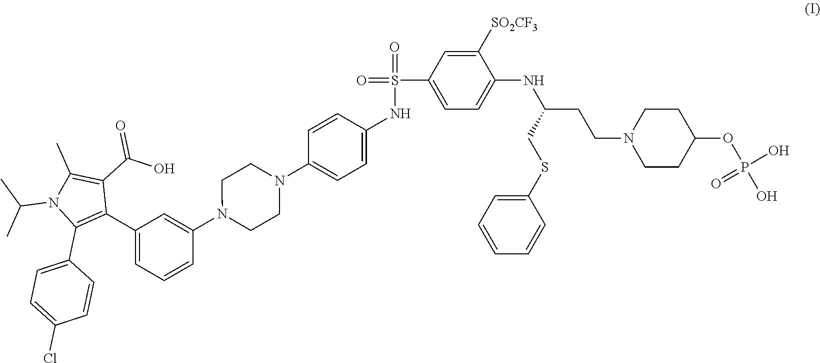

Aspect 23. The crystalline solid of aspect claim 22, wherein meglurine is present in the crystalline solid in a stoichiometric ratio of from 1 to 3.

Aspect 24. The crystalline solid of any one of aspects 22-23, having an x-ray powder diffraction pattern comprising one or more peaks at about 3.8° 2θ; about 4.2° 2θ; about 6.1° 2θ; about 7.4° 2θ; about 8.6° 2θ; about 10.3° 2θ; about 10.9° 2θ; about 12.7° 2θ; about 13.7° 2θ; about 14.4° 2θ; about 15.3° 2θ; about 15.7° 2θ; about 16.5° 2θ; about 17.0° 2θ; about 17.9° 2θ; about 18.5° 2θ; about 19.5° 2θ; about 20.7° 2θ; about 22.2° 2θ; about 22.5° 2θ; about 23.4° 2θ; about 24.8° 2θ; or about 28.2° 20.

Aspect 25. The crystalline solid of any one of aspects 22-24, wherein Form IVa of the crystalline solid meglumine salt of a compound of Formula I is characterized by a 3.5% weight loss step of by thermogravimetric analysis (TGA) between room temperature to 130° C. and a second weight loss step at about 130° C.

Aspect 26. The crystalline solid of any one of aspects 22-25, wherein Form IVa of the crystalline solid meglumine salt of a compound of Formula I exhibits a first endotherm at about 131° C. and a second endotherm at about 139° C. by differential scanning calorimetry (DSC).

Aspect 27. The crystalline solid of any one of aspects 22-26, wherein the crystalline solid is stable at a temperature of from 2° C. to 8° C. for 12 months or more.

Aspect 28. Form IV of a crystalline solid meglumine salt of a compound of Formula I:

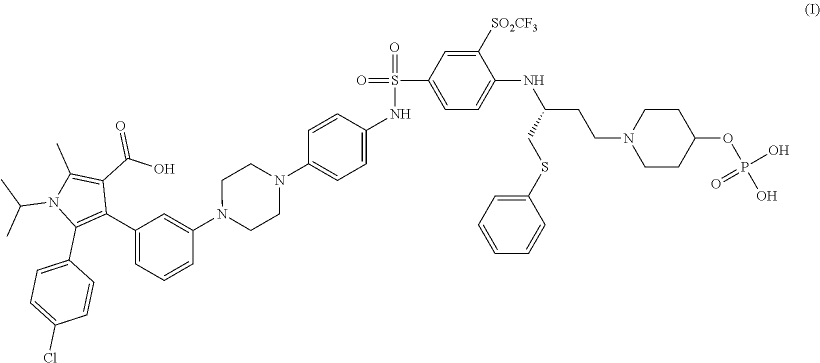

Aspect 29. The crystalline solid of aspect 28, wherein meglumine is present in the crystalline solid in a stoichiometric ratio of from 1 to 3.

Aspect 30. The crystalline solid of any one of aspects 28-29, having an x-ray powder diffraction pattern comprising one or more peaks at about 4.2° 2θ; about 4.6° 2θ; about 7.9° 2θ; about 9.1° 2θ; about 10.4° 2θ; about 13.3° 2θ; about 14.5° 2θ; about 15.8° 20; about 16.8° 2θ; about 17.3° 2θ; about 19.5° 2θ; about 19.6° 2θ; about 20.2° 2θ; or about 27.7° 20.

Aspect 31. The crystalline solid of any one of aspects 28-30, wherein Form IV of the crystalline solid meglumine salt of a compound of Formula I is characterized by a single weight loss step at about 130° C. by thermogravimetric analysis (TGA).

Aspect 32. The crystalline solid of any one of aspects 28-31, wherein Form IV of the crystalline solid meglumine salt of a compound of Formula I exhibits a first endotherm at about 130° C. and a second endotherm at about 143.3° C. by differential scanning calorimetry (DSC).

Aspect 33. The crystalline solid of any one of aspects 28-32, wherein the crystalline solid is stable at a temperature of from 2° C. to 8° C. for 12 months or more.

Aspect 34. Form V of a crystalline solid meglumine salt of a compound of Formula I:

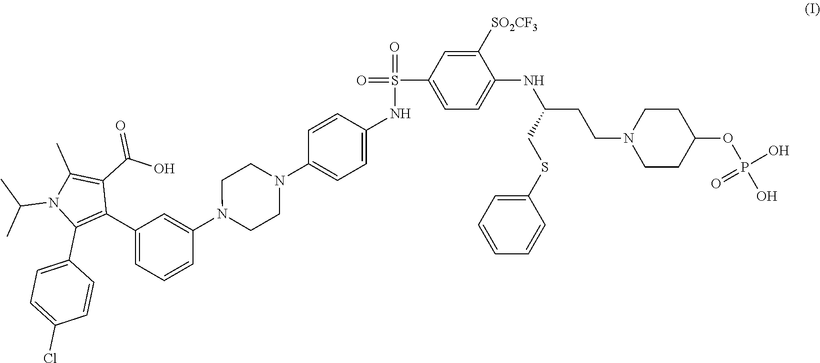

Aspect 35. The crystalline solid of aspect 34, wherein meglumine is present in the crystalline solid in a stoichiometric ratio of from 1 to 3.

Aspect 36. The crystalline solid of any one of aspects 34-35, having an x-ray powder diffraction pattern comprising one or more peaks at about 4.2° 2θ; about 5.4° 2θ; about 7.3° 2θ; about 9.1° 2θ; about 12.2° 2θ; about 12.4° 2θ; about 13.4° 2θ; about 14.5° 2θ; about 16.1° 2θ; about 17.5° 2θ; about 18.1° 2θ; about 18.8° 2θ; about 19.6° 2θ; about 20.4° 2θ; about 21.2° 2θ; about 22.3° 2θ; about 23.0° 2θ; about 27.6° 2θ; or about 29.2° 2θ.

Aspect 37. The crystalline solid of any one of aspects 34-36, wherein Form V of the crystalline solid meglumine salt of a compound of Formula I is characterized by a 1.2% weight loss step of by thermogravimetric analysis (TGA) between room temperature to 130° C. and a second weight loss step at about 130° C.

Aspect 38. The crystalline solid of any one of aspects 34-37, wherein Form V of the crystalline solid meglumine salt of a compound of Formula I exhibits a first endotherm at about 115° C. and a second endotherm at about 143° C. by differential scanning calorimetry (DSC).

Aspect 39. The crystalline solid of any one of aspects 34-38, wherein the crystalline solid is stable at a temperature of from 2° C. to 8° C. for 12 months or more.

Aspect 40. Form VI of a crystalline solid meglumine salt of a compound of Formula I:

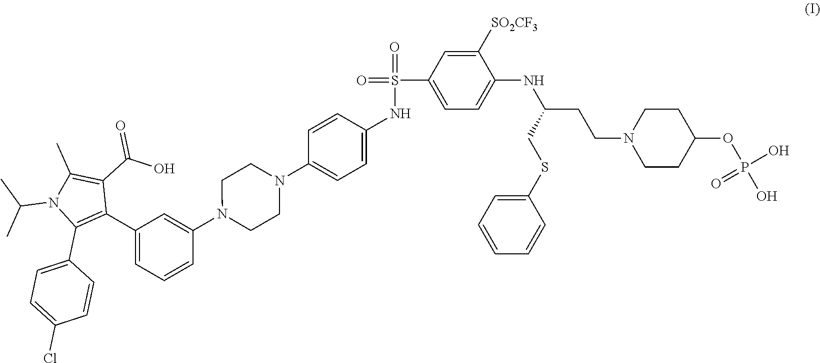

Aspect 41. The crystalline solid of aspect 40, wherein meglumine is present in the crystalline solid in a stoichiometric ratio of from 1 to 3.

Aspect 42. The crystalline solid of any one of aspects 40-41, having an x-ray powder diffraction pattern comprising one or more peaks at about 3.9° 2θ; about 8.5° 2θ; about 8.6° 2θ; about 8.7° 2θ; about 11.3° 2θ; about 12.7° 2θ; about 13.9° 2θ; about 14.5° 2θ; about 15.1° 2θ; about 15.9° 2θ; about 17.6° 2θ; about 17.7° 2θ; about 18.8° 2θ; about 20.0° 2θ; about 20.7° 2θ; about 23.0° 2θ; about 35.1° 2θ; about 36.1° 2θ; or about 36.8° 2θ.

Aspect 43. The crystalline solid of any one of aspects 40-42, wherein Form VI of the crystalline solid meglumine salt of a compound of Formula I is characterized by a 1.0% weight loss step of by thermogravimetric analysis (TGA) between room temperature to 130° C. and a second weight loss step at about 130° C.

Aspect 44. The crystalline solid of any one of aspects 40-43, wherein Form VI of the crystalline solid meglumine salt of a compound of Formula I exhibits a first endotherm at about 110° C. and a second endotherm at about 142° C. by differential scanning calorimetry (DSC).

Aspect 45. The crystalline solid of any one of aspects 28-30, wherein the crystalline solid is stable at a temperature of from 2° C. to 8° C. for 12 months or more.

Aspect 46. A method of making a crystalline solid meglumine salt compound of any one of aspects 1-45, the method comprising: generating a clear solution comprising a meglumine salt of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((triflluoromethyl)sulfonyl)phenyl)sulfonamido)phenyl)piperazin-1-yl)phenyl)-1H-pyrrole-3-carboxylic acid; contacting an aliquot of the clear solution with a seed composition and a solvent composition to generate a first suspension; contacting the first suspension with a second aliquot of the clear solution and a solvent composition to generate a slurry composition; and filtering a crystalline solid meglumine salt of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid from the slurry composition.

Aspect 47. The method of aspect 46, wherein the method comprises: contacting meglumine and (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid in a first solvent composition to generate a first solution comprising solubilized (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid meglumine salt; contacting the first composition with a second solvent composition to generate a clear solution; contacting a first aliquot of the clear solution with a third solvent composition and a seed composition to generate a first suspension; contacting the first suspension with a fourth solvent composition to generate a second suspension; contacting the second suspension with a fifth solvent composition to generate a third suspension; contacting a second aliquot of the clear solution and a sixth solvent composition with the third suspension to generate a slurry precursor composition; contacting the slurry precursor composition with a seventh solvent composition to generate a slurry composition; and filtering a crystalline solid meglumine salt of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid from the slurry composition.

Aspect 48. The method of aspect 47, wherein the first solvent composition comprises two or more polar solvents.

Aspect 49. The method of aspect 48, wherein the first solvent composition comprises a polar aprotic solvent and a polar protic solvent.

Aspect 50. The method of any one of aspects 48-49, wherein the first solvent composition comprises tetrahydrofuran and water.

Aspect 51. The method of aspect 50, wherein the first solvent composition comprises about 9/1 v/v tetrahydrofuran and water.

Aspect 52. The method of any one of aspects 47-51, wherein the second solvent composition comprises a polar solvent.

Aspect 53. The method of aspect 52, wherein the second solvent composition comprises a polar protic solvent.

Aspect 54. The method of aspect 53, wherein the second solvent composition comprises ethanol.

Aspect 55. The method of any one of aspects 47-54, wherein the second solvent composition comprises a polar aprotic solvent.

Aspect 56. The method of aspect claim 55, wherein the second solvent composition comprises ethyl acetate.

Aspect 57. The method of any one of aspects 47-56, wherein contacting the first composition with a second solvent composition comprises contacting the first composition with a polar protic solvent followed by contacting with a polar aprotic solvent.

Aspect 58. The method of aspect 57, wherein contacting the first composition with a second solvent composition comprises contacting the first composition with ethanol followed by contacting with ethyl acetate.

Aspect 59. The method of any one of aspects 47-58, wherein the first aliquot comprises from about 5% to about 15% by volume of the clear solution.

Aspect 60. The method of aspect 59, wherein the first aliquot comprises about 10% by volume of the clear solution.

Aspect 61. The method of aspect 59, wherein the seed composition comprises about 0.9% wt.

Aspect 62. The method of aspect claim 59, wherein the first aliquot comprises from about 7.5% wt to about 10% wt.

Aspect 63. The method of any one of aspects 47-62, wherein the fourth solvent composition comprises a polar protic solvent and a polar aprotic solvent.

Aspect 64. The method of aspect 63, wherein the fourth solvent composition comprises ethanol and ethyl acetate.

Aspect 65. The method of aspect 63, wherein contacting the first suspension with a fourth solvent composition comprises contacting the first suspension with a mixed solvent composition followed by contacting with a polar aprotic solvent.

Aspect 66. The method of aspect 65, wherein contacting the first suspension with a fourth solvent composition comprises contacting the first suspension with an ethanol and ethyl acetate mixed solvent composition followed by contacting with ethyl acetate.

Aspect 67. The method of any one of aspects 47-66, wherein the fifth solvent composition comprises 3 or more solvents.

Aspect 68. The method of aspect 67, wherein the fifth solvent composition comprises tetrahydrofuran, water, ethanol and ethyl acetate.

Aspect 69. The method of any one of aspects 47-68, wherein the sixth solvent composition comprises a polar protic solvent and a polar aprotic solvent.

Aspect 70. The method of aspect 69, wherein the sixth solvent composition comprises ethanol and ethyl acetate.

Aspect 71. The method of any one of aspects 47-70, wherein the seventh solvent composition comprises a polar aprotic solvent.

Aspect 72. The method of aspect 71, wherein the polar aprotic solvent comprises ethyl acetate.

Aspect 73. A composition comprising: a crystalline solid meglumine salt of any one of aspects 1-45; and a pharmaceutically acceptable excipient.

Aspect 74. Use of a crystalline solid meglumine salt of any one of aspects 1-45 in the treatment of a subject.

Aspect 75. Use of a crystalline solid meglumine salt of any one of aspects 1-45 in the treatment of age-related macular degeneration.

Aspect 76. Use of a crystalline solid meglumine salt of any one of aspects 1-45 in the treatment of diabetic macular edema.

Aspect 77. Use of a crystalline solid meglumine salt of any one of aspects 1-45 in the treatment of diabetic retinopathy.

Aspect 78. Use of a crystalline solid meglumine salt of any one of aspects 1-45 in the treatment of a senescence-related condition.

Aspect 79. Use of aspect 76, wherein the condition is osteoarthritis.

Aspect 80. Use of aspect 76, wherein the condition is a pulmonary condition.

Aspect 81. A method comprising administering to a subject in need thereof an amount of a crystalline solid meglumine salt of any one of aspects 1-45.

Aspect 82. A method for treating a subject for an ophthalmic condition, the method comprising administering to the subject an amount of a crystalline solid meglumine salt of any one of aspects 1-45.

Aspect 83. A method for treating a subject for age-related macular degeneration, the method comprising administering to the subject an amount of a crystalline solid meglumine salt of any one of aspects 1-45.

Aspect 84. A method for treating a subject for diabetic macular edema, the method comprising administering to the subject an amount of a crystalline solid meglumine salt of any one of aspects 1-45.

Aspect 85. A method for treating a subject for diabetic retinopathy, the method comprising administering to the subject an amount of a crystalline solid meglumine salt of any one of aspects 1-45.

Aspect 86. A method for treating a subject for a senescence-related condition, the method comprising administering to the subject an amount of a crystalline solid meglumine salt of any one of aspects 1-45.

Aspect 87. The method of aspect 86, wherein the condition is osteoarthritis.

Aspect 88. The method of aspect 86, wherein the condition is a pulmonary condition.

Aspect 89. Use of a crystalline solid meglumine salt of any one of aspects 1-45 in the manufacture of a medicament for treating a subject.

Aspect 90. Use of a crystalline solid meglumine salt of any one of aspects 1-45 in the manufacture of a medicament for treating age-related macular degeneration in a subject.

Aspect 91. Use of a crystalline solid meglumine salt of any one of aspects 1-45 in the manufacture of a medicament for treating diabetic macular edema in a subject.

Aspect 92. Use of a crystalline solid meglumine salt of any one of aspects 1-45 in the manufacture of a medicament for treating senescence-related condition in a subject.

Aspect 93. Use of aspect 92, wherein the condition is osteoarthritis.

Aspect 94. Use of aspect 92, wherein the condition is a pulmonary condition.

### BRIEF DESCRIPTION OF THE FIGURES

The invention may be best understood from the following detailed description when read in conjunction with the accompanying drawings. Included in the drawings are the following figures:

FIG. 1 depicts an X-ray Powder Diffraction (XRPD) of Forms I-VI and IVA of the crystalline solid meglumine salts of the subject compounds.

FIG. 2A depicts a Polarized Light Microscope (PLM) image of crystals of Form 1. FIG. 2B depicts Thermogravimetric Analysis (TGA) and Differential Scanning Calorimetry (DSC) analysis of crystals of Form I.

FIG. 3 depicts Dynamic Vapor Sorption (DVS) analysis of crystals of Form I.

FIG. 4 depicts an XRPD of crystals of Form I.

FIG. 5A depicts a PLM image of crystals of Form II. FIG. 5B depicts TGA and DSC analysis of crystals of Form II.

FIG. 6 depicts an XRPD of crystals of Form II.

FIG. 7A depicts a PLM image of crystals of Form III. FIG. 7B depicts TGA and DSC analysis of crystals of Form III.

FIG. 8 depicts DVS analysis of crystals of Form III.

FIG. 9 depicts an XRPD of crystals of Form III.

FIG. 10A depicts a PLM image of crystals of Form IV. FIG. 10B depicts TGA and DSC analysis of crystals of Form IV.

FIG. 11 depicts DVS analysis of crystals of Form IV.

FIG. 12 depicts an XRPD of crystals of Form IV.

FIG. 13A depicts a PLM image of crystals of Form V. FIG. 13B depicts TGA and DSC analysis of crystals of Form V.

FIG. 14 depicts DVS analysis of crystals of Form V.

FIG. 15 depicts an XRPD of crystals of Form V.

FIG. 16A depicts a PLM image of crystals of Form 6. FIG. 16B depicts TGA and DSC analysis of crystals of Form VI.

FIG. 17 depicts DVS analysis of crystals of Form VI.

FIG. 18 depicts an XRPD of crystals of Form VI.

FIG. 19 depicts an XRPD of crystals of Forms II-VI (at Day 0 of a stability test) and after being subjected to temperatures of 60° C. for 7 days.

FIG. 20 depicts the stability of a crystalline solid meglumine salt of the subject compounds characterized over 12 months.

FIG. 21 depicts an XRPD of crystals of Forms IV and V. FIG. 21 shows that crystals of Form V convert to Form IV when subjected to a stability study at 40° C./75% RH.

FIG. 22 depicts the change to the XRPD of crystals of Forms III, V and VI when heated to 130° C. Under thermal treatment by heating crystals of Forms III, V and VI with a ramping rate of 5° C./min, the crystals of Forms III, V and VI converted to Form IV upon heating.

### DETAILED DESCRIPTION

Aspects of the disclosure include crystalline solid meglumine salts of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid. Pharmaceutical compositions having one or more of the subject crystalline solid meglumine salt compounds and methods for administering the crystalline solid meglumine salt compounds to a subject are also described. Methods for preparing the subject crystalline sold meglumine salt compounds are also provided.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed, to the extent that such combinations embrace subject matter that are, for example, compounds that are stable compounds (i.e., compounds that can be made, isolated, characterized, and tested for biological activity). In addition, all sub-combinations of the various embodiments and elements thereof (e.g., elements of the chemical groups listed in the embodiments describing such variables) are also specifically embraced by the present invention and are disclosed herein just as if each and every such sub-combination was individually and explicitly disclosed herein.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, methods and materials of interest are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

The nomenclature used herein to name the subject compounds is illustrated in the Examples herein. When possible, this nomenclature has generally been derived using the commercially-available AutoNom software (MDL, San Leandro, Calif.).

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Compounds as described herein can be purified by any of the means known in the art, including chromatographic means, such as high performance liquid chromatography (HPLC), preparative thin layer chromatography, flash column chromatography and ion exchange chromatography. Any suitable stationary phase can be used, including normal and reversed phases as well as ionic resins. See, e.g., Introduction to Modern Liquid Chromatography, 2nd Edition, ed. L. R. Snyder and J. J. Kirkland, John Wiley and Sons, 1979; and Thin Layer Chromatography, ed E. Stahl, Springer-Verlag, New York, 1969.

During any of the processes for preparation of the compounds of the present disclosure, it may be necessary and/or desirable to protect sensitive or reactive groups on any of the molecules concerned. This can be achieved by means of conventional protecting groups as described in standard works, such as T. W. Greene and P. G. M. Wuts, “Protective Groups in Organic Synthesis”, Fourth edition, Wiley, New York 2006. The protecting groups can be removed at a convenient subsequent stage using methods known from the art.

The compounds described herein can contain one or more chiral centers and/or double bonds and therefore, can exist as stereoisomers, such as double-bond isomers (i.e., geometric isomers), enantiomers or diastereomers. Accordingly, all possible enantiomers and stereoisomers of the compounds including the stereoisomerically pure form (e.g., geometrically pure, enantiomerically pure or diastereomerically pure) and enantiomeric and stereoisomeric mixtures are included in the description of the compounds herein. Enantiomeric and stereoisomeric mixtures can be resolved into their component enantiomers or stereoisomers using separation techniques or chiral synthesis techniques well known to the skilled artisan. The compounds can also exist in several tautomeric forms including the enol form, the keto form and mixtures thereof. Accordingly, the chemical structures depicted herein encompass all possible tautomeric forms of the illustrated compounds. The compounds described also include isotopically labeled compounds where one or more atoms have an atomic mass different from the atomic mass conventionally found in nature. Examples of isotopes that can be incorporated into the compounds disclosed herein include, but are not limited to, ²H, ³H, ¹¹C, ¹³C, ¹⁴C, ¹⁵N, ¹⁸O, ¹⁷O, etc. Compounds can exist in unsolvated forms as well as solvated forms, including hydrated forms. In general, compounds can be hydrated or solvated. Certain compounds can exist in multiple crystalline or amorphous forms. In general, all physical forms are equivalent for the uses contemplated herein and are intended to be within the scope of the present disclosure.

Aspects of the disclosure include crystalline solid meglumine salts of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid. The term “crystalline” is used herein in its conventional sense to refer to a solid material where the molecules that form the solid are arranged in a highly ordered microscopic geometric configuration (e.g., form an ordered lattice-type structure) that extends in three dimensions. In embodiments, crystalline solids described herein are not amorphous, which are characterized by undefined structural order and microscopic configurations that lack a regular geometric arrangement in three dimensions.

As described herein, the compound (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid is a compound of Formula I:

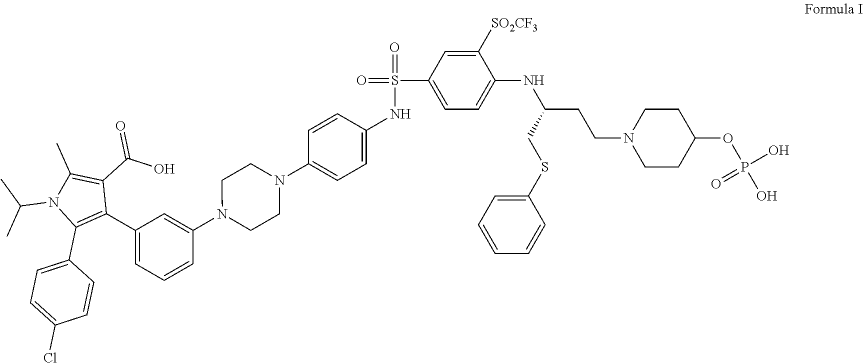

The compound meglumine refers to the amino sugar derived from glucose, (2R,3R,4R,5S)-6-(Methylamino)hexane-1,2,3,4,5-pentol, having a structure:

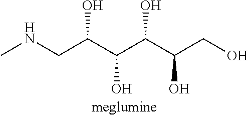

In some embodiments, meglumine is present in the subject crystalline solids in a stoichiometric ratio with (R)-5-(4-chlorophenyl)-1-isopropyl-2-methyl-4-(3-(4-(4-((4-((I-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid of from 1:10, such as from 1:9, such as from 1:8, such as from 1:7, such as from 1:6, such as from 1:5, such as from 1:4, such as from 1:3, such as from 1:2 and including from 1:1. In other embodiments, meglumine is present in the subject crystalline solids in a stoichiometric ratio with (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid of from 10:1, such as from 9:1, such as from 8:1, such as from 7:1, such as from 6:1, such as from 5:1, such as from 4:1, such as from 3:1 and including from 2:1.

The present disclosure uses the term “Form” to identify different crystalline forms of crystalline (R)-5-(4-chlorophenyl)-1-isopropyl-2-methyl-4-(3-(4-(4-((4-((I-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid meglumine salt or liquid crystalline forms. The differences in the forms can be seen by structure, such as x-ray powder diffraction; properties such as hygroscopicity or thermal behaviors; and/or both. The use of the term “Form I” means crystalline (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid meglumine salt of Form 1. Likewise, “Form II” means crystalline (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid meglumine salt of Form II. Similarly, Form III, Form IV, Form IVa, Form V and Form VI mean crystalline (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid meglumine salt of Form III, Form IV, Form IVa, Form V and Form VI, respectively.

In embodiments, the crystalline solid meglumine salts of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifiuoromethyl)sulfonyl)phenyl)sulfonamido)phenyl)piperazin-1-yl)phenyl)-1H-pyrrole-3-carboxylic acid has a polymorph purity (i.e., is present as the polymorph as evidenced by X-ray powder diffraction (XRPD) analysis, thermogravimetric analysis (TGA) and differential scanning calorimetry (DSC) analysis, described in greater detail below) that is 90% or greater, such as 95% or greater, such as 97% or greater, such as 99% or greater and including 99.9% or greater. In some embodiments, the polymorph form of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid meglumine salt described herein is present in the crystalline solid in 100% purity. In some embodiments, the crystalline solid meglumine salts of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl-1H-pyrrole-3-carboxylic acid provided herein exhibits improved solubility and reactivity as compared to other salts (e.g., sodium, potassium) of crystalline (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid and amorphous (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluororethyl)sulfonyl)phenyl)sulfonamido)phenyl)piperazin-1-yl)phenyl)-1H-pyrrole-3-carboxylic acid. In some embodiments, the subject crystalline solid meglumine salts are stable at a temperature of from 2° C. to 8° C. for 3 months or more, such as 6 months or more, such as 9 months or more, such as 12 months or more, such as 18 months or more, such as 24 months or more, such as 36 months or more, such as 48 months or more and including being stable at a temperature from 2° C. to 8° C. for 60 months or more.

The crystalline solid meglumine salts of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid can be analyzed by x-ray powder diffraction. An x-ray powder diffraction pattern is an x-y graph with ° 20 (diffraction angle) on the x-axis and intensity on the y-axis. The pattern contains peaks which may be used to characterize the subject crystalline solid meglumine salts. The peaks are usually represented and referred to by their position on the x-axis rather than the intensity of peaks on the y-axis because peak intensity can be particularly sensitive to sample orientation (see Pharmaceutical Analysis, Lee & Web, pp. 255-257 (2003)). Thus, intensity is not typically used to characterize solid forms.

The data from x-ray powder diffraction may be used in multiple ways to characterize crystalline forms. For example, the entire x-ray powder diffraction pattern output from a diffractometer may be used to characterize crystalline solid meglumine salts of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid. A smaller subset of such data, however, may also be, and typically is, suitable for characterizing the crystalline solid meglumine salts of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid. For example, a collection of one or more peaks from such a pattern may be used to characterize the crystalline solid meglumine salts of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid. In the present application, all reported peak values are in ° 20 with Cu-Kα radiation. Indeed, often even a single x-ray powder diffraction peak may be used to characterize such a crystalline form. When the crystalline solid meglumine salts of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid herein are characterized by “one or more peaks” of an x-ray powder diffraction pattern and such peaks are listed, what is generally meant is that any combination of the peaks listed may be used to characterize the crystalline solid meglumine salt of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)suilfonyl)phenyl)sulfonamido)phenyl)piperazin-1-yl)phenyl)-H-pyrrole-3-carboxylic acid. Further, the fact that other peaks are present in the x-ray powder diffraction pattern, generally does not negate or otherwise limit that characterization.

In addition to the variability in peak intensity, there may also be variability in the position of peaks on the x-axis. This variability can, however, typically be accounted for when reporting the positions of peaks for purposes of characterization. Such variability in the position of peaks along the x-axis may derive from several sources (e.g., sample preparation, particle size, moisture content, solvent content, instrument parameters, data analysis software, and sample orientation). For example, samples of the same crystalline material prepared under different conditions may yield slightly different diffractograms, and different x-ray instruments may operate using different parameters and these may lead to slightly different diffraction patterns from the same crystalline solid.

Due to such sources of variability, it is common to recite x-ray diffraction peaks using the word “about” prior to the peak value in ° 2θ. For purposes of data reported herein, that value is generally ±0.1° 2θ. This generally means that on a well-maintained instrument one would expect the variability in peak measurement to be ±0.1° 2θ. Unless specified otherwise, x-ray powder diffraction peaks cited herein are generally reported with this variability of ±0.1° 2θ and are generally intended to be reported with such a variability whenever disclosed herein whether the word “about” is present or not, however, variability may, in some instances, be as high as ±0.2° 20 or even higher depending on instrumentation conditions.

Aspects of the present disclosure include Form 1 of a crystalline solid meglumine salt of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid. In embodiments, the polymorph Form I of crystalline solid (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid meglumine salt exhibits an X-ray powder diffraction (XRPD) pattern having one or more peaks at about 4.3° 2θ; about 6.1° 2θ; about 8.1° 2θ; about 8.6° 2θ; about 9.0° 2θ; about 10.1° 2θ; about 11.3° 2θ; about 12.2° 2θ; about 15.2° 2θ; about 16.2° 2θ; about 17.3° 2θ; about 18.2° 2θ; about 18.9° 2θ; about 19.3° 2θ; about 19.8° 2θ; about 20.7° 2θ; about 21.6° 2θ; about 22.1° 2θ; about 23.0° 2θ; about 24.2° 2θ; about 25.2° 2θ; about 25.5° 2θ; about 26.1° 2θ; about 27.1° 2θ; about 29.5° 2θ; or about 3.2.6° 2θ. For a given crystal form, the relative intensity of a diffraction peak may vary due to orientation of the crystal relative to the x-rays such as from crystalline morphology. In embodiments, the intensity of x-ray powder diffraction peak in 2θ may vary from crystal to crystal, but the characteristic peak positions for the polymorph form will remain the same. The polymorph Form I of the crystalline solid meglumine salt of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid provided herein is, in some instances, characterized by a 0.9% weight loss step of by thermogravimetric analysis (TGA) between room temperature to 130° C. and a second weight loss step at about 130° C. Differential scanning calorimetry (DSC) measures the transition temperature of a crystalline solid when the crystal absorbs or releases heat due to a change in its structure or due to melting. DSC provides for distinguishing between different crystalline forms (e.g., different polymorphs). Different crystal forms may be identified according to their different characteristic transition temperatures. In some embodiments, the polymorph Form I of the crystalline solid meglumine salt of (R)-5-(4-chlorophenyl)-1-isopropyl-2-methyl-4-(3-(4-(4-((4-((1-(phenylthio)-4-(4-((phosphonooxy)methyl)piperidin-1H-yl)butan-2-yl)amino)-3-((trifluoromethyl)sulfonyl)phenyl)sulfonamido)phenyl)piperazin-1-yl)phenyl)-1H-pyrrole-3-carboxylic acid provided herein exhibits a first endotherm at about 84° C. and a second endotherm at about 147° C. by differential scanning calorimetry (DSC).

Aspects of the present disclosure include Form II of a crystalline solid meglumine salt of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifliuoromethyl)sulfonyl)phenyl)sulfonamido)phenyl)piperazin-1-yl)phenyl)-1H-pyrrole-3-carboxylic acid. In embodiments, the polymorph Form II of crystalline solid (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid meglumine salt exhibits an X-ray powder diffraction (XRPD) pattern having one or more peaks at about 3.8° 2θ; about 7.3° 2θ; about 8.3° 2θ; about 8.8° 2θ; about 13.7° 2θ; about 15.2° 2θ; about 15.4° 2θ; about 16.6° 2θ; about 17.7° 2θ; about 18.8° 2θ; about 20.0° 2θ; about 22.1° 2θ; or about 23.9° 2θ. The polymorph Form II of the crystalline solid meglumine salt of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1-pyrrole-3-carboxylic acid provided herein is, in some instances, characterized by a 2.0% weight loss step of by thermogravimetric analysis (TGA) between room temperature to 130° C. and a second weight loss step at about 130° C. In some embodiments, the polymorph Form II of the crystalline solid meglumine salt of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid provided herein exhibits an endotherm at about 136° C. by differential scanning calorimetry (DSC).

Aspects of the present disclosure include Form III of a crystalline solid meglumine salt of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid. In embodiments, the polymorph Form III of crystalline solid (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid meglumine salt exhibits an X-ray powder diffraction (XRPD) pattern having one or more peaks at about 3.9° 2θ; about 4.3° 2θ; about 6.1° 2θ; about 7.5° 2θ; about 7.7° 2θ; about 8.7° 2θ; about 10.4° 2θ; about 11.3° 2θ; about 11.5° 2θ; about 12.5° 2θ; about 13.9° 2θ; about 14.7° 2θ; about 15.2° 2θ; about 15.9° 2θ; about 17.7° 2θ; about 18.0° 2θ; about 18.8° 2θ; about 20.2° 2θ; about 21.7° 2θ; about 23.0° 2θ; or about 25.8° 2θ. The polymorph Form III of the crystalline solid meglumine salt of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1 l-yl)phenyl)-1H-pyrrole-3-carboxylic acid provided herein is, in some instances, characterized by a 0.9% weight loss step of by thermogravimetric analysis (TGA) between room temperature to 130° C. and a second weight loss step at about 130° C. In some embodiments, the polymorph Form III of the crystalline solid meglumine salt of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid provided herein exhibits a first endotherm at about 113° C. and a second endotherm at about 142° C. by differential scanning calorimetry (DSC).

Aspects of the present disclosure include Form IVa of a crystalline solid meglumine salt of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid. In embodiments, the polymorph Form IVa of crystalline solid (R)-5-(4-chiorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid meglumine salt exhibits an X-ray powder diffraction (XRPD) pattern having one or more peaks at about 3.8° 2θ; about 4.2° 2θ; about 6.1° 2θ; about 7.4° 2θ; about 8.6° 2θ; about 10.3° 2θ; about 10.9° 2θ; about 12.7° 2θ; about 13.7° 2θ; about 14.4° 2θ; about 15.3° 2θ; about 15.7° 2θ; about 16.5° 2θ; about 17.0° 2θ; about 17.9° 2θ; about 18.5° 2θ; about 19.5° 2θ; about 20.7° 2θ; about 22.2° 2θ; about 22.5° 2θ; about 23.4° 2θ; about 24.8° 2θ; or about 28.2° 2θ. The polymorph Form IVa of the crystalline solid meglumine salt of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid provided herein is, in some instances, characterized by a 3.5% weight loss step of by thermogravimetric analysis (TGA) between room temperature to 130° C. and a second weight loss step at about 130° C. In some embodiments, the polymorph Form IVa of the crystalline solid meglumine salt of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid provided herein exhibits a first endotherm at about 113° C. and a second endotherm at about 142° C. by differential scanning calorimetry (DSC).

Aspects of the present disclosure include Form IV of a crystalline solid meglumine salt of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid. In embodiments, the polymorph Form IV of crystalline solid (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid meglumine salt exhibits an X-ray powder diffraction (XRPD) pattern having one or more peaks at about 4.2° 2θ; about 4.6° 2θ; about 7.9° 2θ; about 9.1° 2θ; about 10.4° 2θ; about 13.3° 2θ; about 14.5° 2θ; about 15.8° 2θ; about 16.8° 2θ; about 17.3° 2θ; about 19.5° 2θ; about 19.6° 2θ; about 20.2° 2θ; or about 27.7° 2θ. The polymorph Form IV of the crystalline solid meglumine salt of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid provided herein is, in some instances, characterized by a single weight loss step at about 130° C. by thermogravimetric analysis (TGA). In some embodiments, the polymorph Form IV of the crystalline solid meglumine salt of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid provided herein exhibits a first endotherm at about 130° C. and a second endotherm at about 143.3° C. by differential scanning calorimetry (DSC).

Aspects of the present disclosure include Form V of a crystalline solid meglumine salt of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid. In embodiments, the polymorph Form V of crystalline solid (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid meglumine salt exhibits an X-ray powder diffraction (XRPD) pattern having one or more peaks at about 4.2° 2θ; about 5.4° 2θ; about 73° 2θ; about 9.1° 2θ; about 12.2° 2θ; about 12.4° 2θ; about 13.4° 2θ; about 14.5° 2θ; about 16.1° 2θ; about 17.5° 2θ; about 18.1° 2θ; about 18.8° 2θ; about 19.6° 26; about 20.4° 2θ; about 21.2° 2θ; about 22.3° 2θ; about 23.0° 2θ; about 27.6° 2θ; or about 29.2° 2θ. The polymorph Form V of the crystalline solid meglumine salt of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid provided herein is, in some instances, characterized by a 1.2% weight loss step of by thermogravimetric analysis (TGA) between room temperature to 130° C. and a second weight loss step at about 130° C. In some embodiments, the polymorph Form V of the crystalline solid meglumine salt of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid provided herein exhibits a first endotherm at about 115° C. and a second endotherm at about 143° C. by differential scanning calorimetry (DSC).

Aspects of the present disclosure include Form VI of a crystalline solid meglumine salt of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid. In embodiments, the polymorph Form VI of crystalline solid (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid meglumine salt exhibits an X-ray powder diffraction (XRPD) pattern having one or more peaks at about 3.9° 2θ; about 8.5° 2θ; about 8.6° 2θ; about 8.7° 2θ; about 11.3° 2θ; about 12.7° 2θ; about 13.9° 2θ; about 14.5° 2θ; about 15.1° 2θ; about 15.9° 2θ; about 17.6° 2θ; about 17.7° 2θ; about 18.8° 2θ; about 20.0° 2θ; about 20.7° 2θ; about 23.0° 2θ; about 35.1° 2θ; about 36.1° 2θ; or about 36.8° 2θ. The polymorph Form VI of the crystalline solid meglumine salt of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid provided herein is, in some instances, characterized by a 1.0% weight loss step of by thermogravimetric analysis (TGA) between room temperature to 130° C. and a second weight loss step at about 130° C. In some embodiments, the polymorph Form VI of the crystalline solid meglumine salt of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid provided herein exhibits a first endotherm at about 110° C. and a second endotherm at about 142° C. by differential scanning calorimetry (DSC).

Methods for preparing crystalline solid meglumine salts of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)suilfonyl)phenyl)sulfonamido)phenyl)piperazin-1-yl)phenyl)-1H-pyrrole-3-carboxylic acid are also provided. In practicing methods according to certain embodiments, a clear solution of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifiuoromethyl)suilfonyl)phenyl)sulfonamido)phenyl)piperazin-1-yl)phenyl)-1H-pyrrole-3-carboxylic acid meglumine salt is generated by dissolving free acid and meglumine in a solvent. In some embodiments, the solvent includes a polar protic solvent. In other embodiments, the solvent includes a polar aprotic solvent. In still other embodiments, the solvent is mixture of a polar protic solvent and a polar aprotic solvent. Polar protic solvents of interest may include, but are not limited to, ammonia, t-butanol, n-propanol, ethanol, methanol, acetic acid, water. Polar aprotic solvents of interest may include tetrahydrofuran, methyltetrahydrofuran, dimethylformamide (DMF), acetone, dimethylsulfoxide (DMSO) and acetonitrile, dichloromethane, ethyl acetate and combinations thereof. In some instances, the solvent a combination of a polar aprotic solvent and a polar protic solvent. Where the solvent is a combination of a polar aprotic solvent and a polar protic solvent, the volume ratio of the polar aprotic solvent to polar protic solvent may be range from 100:1 to 1:1, such as from 90:1 to 1:1, such as from 80:1 to 1:1, such as from 70:1 to 1:1, such as from 60:1 to 1:1, such as from 50:1 to 1:1, such as from 40:1 to 1:1, such as from 30:1 to 1:1, such as from 20:1 to 1:1, such as from 10:1 to 1:1, such as 10:1 or 9:1 or 8:1 or 7:1 or 6:1 or 5:1 or 4:1 or 3:1 or 2:1. In other embodiments, the volume ratio of the polar aprotic solvent to polar protic solvent ranges from 1:100 to 1:1, such as from 1:90 to 1:1, such as from 1:80 to 1:1, such as from 1:70 to 1:1, such as from 1:60 to 1:1, such as from 1:50 to 1:1, such as from 1:40 to 1:1, such as from 1:30 to 1:1, such as from 1:20 to 1:1, such as from 1:10 to 1:1, such as 1:9 or 1:8 or 1:7 or 1:6 or 1:5 or 1:4 or 1:3 or 1:2 and including 1:1. In certain instances, the clear solution of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid meglumine salt is generated by dissolving free acid and meglumine in a solvent that includes tetrahydrofuran and water, such as in a volume ratio of 9:1 v/v.

In some embodiments, generating the clear solution includes contacting the dissolved (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid meglumine salt (e.g., in THF/water) with a second solvent. Solvents of interest may include, but are not limited to, ammonia, t-butanol, n-propanol, ethanol, methanol, acetic acid, water, tetrahydrofuran, methyltetrahydrofuran, dichloromethane, isopropylacetate, ethyl acetate, 1,2-dichloroethane (DCE), dimethylformamide (DMF), acetone, dimethylacetamide, dimethylsulfoxide (DMSO), acetonitrile, toluene, 2-methylbutan-2-ol (tAmOH) and N-methyl-2-pyrrolidone (NMP) and combinations thereof. In certain embodiments, the solvent is a polar protic solvent. In certain instances, the solvent is ethanol. In certain instances, methods further include contacting the composition with a third solvent. Solvents of interest may include, but are not limited to, ammonia, t-butanol, n-propanol, ethanol, methanol, acetic acid, water, tetrahydrofuran, methyltetrahydrofuran, dichloromethane, isopropylacetate, ethyl acetate, 1,2-dichloroethane (DCE), dimethylformamide (DMF), acetone, dimethylacetamide, dimethylsulfoxide (DMSO), acetonitrile, toluene, 2-methylbutan-2-ol (tAmOH) and N-methyl-2-pyrrolidone (NMP) and combinations thereof. In certain embodiments, the solvent is a polar aprotic solvent. In certain instances, the third solvent is ethyl acetate. In the subject methods, an aliquot of the clear solution is contacted with a seed composition of the (R)-5-(4-chlorophenyl)-1-isopropyl-2-methyl-4-(3-(4-(4-((4-((1-(phenylthio)-4-(4-((phosphonooxy)methyl)piperidin-1H-yl)butan-2-yl)amino)-3-((trifluoromethyl)sulfonyl)phenyl)sulfonamido)phenyl)piperazin-1-yl)phenyl)-1H-pyrrole-3-carboxylic acid meglumine salt. The aliquot may be 0.1% by weight or more of the clear solution, such as 0.5% by weight or more, such 1.0% by weight or more, such as 2.0% by weight or more, such as 3.0% by weight or more, such as 4.0% by weight or more, such as 5.0% by weight or more, such as 6.0% by weight or more, such as 7.0% by weight or more, such as 8.0% by weight or more, such as 9.0% by weight or more and including 10% by weight or more. In certain instances, the aliquot ranges from 0.1% to 25% by weight of the clear solution, such as from 0.2% to 20%, such as from 0.3% to 15%, such as from 0.4% to 14%, such as from 0.5% to 13%, such as from 0.6% to 12%, such as from 0.7% to 11% and including from 0.8% to 10% by weight of the clear solution. In certain embodiments, the aliquot is about 10% by weight of the clear solution.

In some embodiments, the seed composition is contacted with the clear solution and a solvent. Solvents of interest may include, but are not limited to, ammonia, t-butanol, n-propanol, ethanol, methanol, acetic acid, water, tetrahydrofuran, methyltetrahydrofuran, dichloromethane, isopropylacetate, ethyl acetate, 1,2-dichloroethane (DCE), dimethylformamide (DMF), acetone, dimethylacetamide, dimethylsulfoxide (DMSO), acetonitrile, toluene, 2-methylbutan-2-ol (tAmOH) and N-methyl-2-pyrrolidone (NMP) and combinations thereof. In some embodiments, the solvent is a mixture of 2 or more solvents, such as 3 or more solvents, such as 4 or more solvents, such as 5 or more solvents and including a mixture of 6 or more solvents. In certain embodiments, the seed composition is contacted with the aliquot of clear solution and a solvent mixture that includes tetrahydrofuran, water, ethanol and ethyl acetate. In certain instances, the seed composition is contacted with the aliquot of clear solution and a solvent mixture that includes 9/1 v/v tetrahydrofuran/water:ethanol:ethyl acetate (2/1/2 v/v/v). In these embodiments, the seed suspension may be 0.5% wt. or more, such as 0.6% wt. or more, such as 0.7% wt. or more, such as 0.8% wt. or more, such as 0.9% wt. or more, such as 1.0% wt. or more, such as 1.5% wt. or more, such as 2.0% wt. or more, such as 3.0% wt. or more, such as 4.0% wt. or more, such as 5.0% wt. or more, such as 6.0% wt. or more, such as 7.0% wt. or more, such as 8.0% wt. or more, such as 9.0% wt. or more, such as 10% wt. or more, such as 15% wt. or more and including 20% wt. or more. In certain embodiments, the seed composition is a 0.9% wt. seed composition. In embodiments, contacting the aliquot of clear solution with the seed composition and solvent generates a first suspension.

The first suspension is contacted with a solvent and slurried. Solvents of interest may include, but are not limited to, ammonia, t-butanol, n-propanol, ethanol, methanol, acetic acid, water, tetrahydrofuran, methyltetrahydrofuran, dichloromethane, isopropylacetate, ethyl acetate, 1,2-dichloroethane (DCE), dimethylformamide (DMF), acetone, dimethylacetamide, dimethylsulfoxide (DMSO), acetonitrile, toluene, 2-methylbutan-2-ol (tAmOH) and N-methyl-2-pyrrolidone (NMP) and combinations thereof. In some embodiments, the solvent is a mixture of 2 or more solvents, such as 3 or more solvents, such as 4 or more solvents, such as 5 or more solvents and including a mixture of 6 or more solvents. For example, the first suspension may be contacted with a mixture of ethanol and ethyl acetate.

In certain instances, methods include contacting the first suspension with a first solvent and slurried for a first predetermined period of time, followed by contacting with a second solvent and slurried for a second predetermined period of time. In these embodiments, the second solvent may be ammonia, t-butanol, n-propanol, ethanol, methanol, acetic acid, water, tetrahydrofuran, methyltetrahydrofuran, dichloromethane, isopropylacetate, ethyl acetate, 1,2-dichloroethane (DCE), dimethylformamide (DMF), acetone, dimethylacetamide, dimethylsulfoxide (DMSO), acetonitrile, toluene, 2-methylbutan-2-ol (tAmOH) and N-methyl-2-pyrrolidone (NMP) or a combination thereof. For example, the second solvent may be ethyl acetate. For example, methods may include contacting the first suspension with a solvent mixture of ethanol/ethyl acetate and slurried for a first predetermined period of time followed by contacting with ethyl acetate and slurried for a second predetermined period of time. In these embodiments, the first predetermined period of time and the second predetermined period of time may independently be 1 minute or more, such as 2 minutes or more, such as 3 minutes or more, such as 4 minutes or more, such as 5 minutes or more, such as 10 minutes or more, such as 15 minutes or more, such as 30 minutes or more, such as 45 minutes or more, such as 60 minutes or more, such as 2 hours or more, such as 3 hours or more, such as 4 hours or more, such as 8 hours or more, such as 12 hours or more and including 16 hours or more.

In some embodiments, the slurried suspension composition is further contacted with a solvent composition. In these embodiments, the solvent composition may include one or more of ammonia, t-butanol, n-propanol, ethanol, methanol, acetic acid, water, tetrahydrofuran, methyltetrahydrofuran, dichloromethane, isopropylacetate, ethyl acetate, 1,2-dichloroethane (DCE), dimethylformamide (DMF), acetone, dimethylacetamide, dimethylsulfoxide (DMSO), acetonitrile, toluene, 2-methylbutan-2-ol (tAmOH) and N-methyl-2-pyrrolidone (NMP) or a combination thereof. In certain embodiments, the slurried suspension composition is contacted with a solvent mixture that includes tetrahydrofuran, water, ethanol and ethyl acetate. In certain instances, the slurried suspension composition is contacted with a solvent mixture that includes 9/1 v/v tetrahydrofuran/water:ethanol:ethyl acetate (2/1/2 v/v/v).

The suspension (with added solvent composition) is contacted with a second aliquot of the clear solution and a solvent composition and slurried. The second aliquot may be 10% by weight or more of the clear solution, such as 20% by weight or more, such 30% by weight or more, such as 40% by weight or more, such as 50% by weight or more, such as 60% by weight or more, such as 70% by weight or more, such as 75% by weight or more, such as 80% by weight or more, such as 85% by weight or more and including 90% by weight or more. In certain instances, the aliquot ranges from 10% to 90% by weight of the clear solution, such as from 11% to 89% by weight, such as from 12% to 88% by weight, such as from 13% to 87% by weight, such as from 14% to 86% by weight, such as from 15% to 85% by weight, such as from 16% to 84% by weight, such as from 17% to 83% by weight, such as from 18% to 82% by weight, such as from 19% to 81% by weight and including from 20% to 80% by weight of the clear solution. In certain embodiments, the second aliquot is about 90% by weight of the clear solution. Solvents of interest may include, but are not limited to, ammonia, t-butanol, n-propanol, ethanol, methanol, acetic acid, water, tetrahydrofuran, methyltetrahydrofuran, dichloromethane, isopropylacetate, ethyl acetate, 1,2-dichloroethane (DCE), dimethylformamide (DMF), acetone, dimethylacetamide, dimethylsulfoxide (DMSO), acetonitrile, toluene, 2-methylbutan-2-ol (tAmOH) and N-methyl-2-pyrrolidone (NMP) and combinations thereof. In some embodiments, the solvent is a mixture of 2 or more solvents, such as 3 or more solvents, such as 4 or more solvents, such as 5 or more solvents and including a mixture of 6 or more solvents. For example, the suspension with second aliquot of clear solution may be contacted with a mixture of ethanol and ethyl acetate.

In certain instances, methods include contacting the suspension and second aliquot of clear solution with a first solvent and slurried for a first predetermined period of time, followed by contacting with a second solvent and slurried for a second predetermined period of time. In these embodiments, the second solvent may be ammonia, t-butanol, n-propanol, ethanol, methanol, acetic acid, water, tetrahydrofuran, methyltetrahydrofuran, dichloromethane, isopropylacetate, ethyl acetate, 1,2-dichloroethane (DCE), dimethylformamide (DMF), acetone, dimethylacetamide, dimethylsulfoxide (DMSO), acetonitrile, toluene, 2-methylbutan-2-ol (tAmOH) and N-methyl-2-pyrrolidone (NMP) or a combination thereof. For example, the second solvent may be ethyl acetate. For example, methods may include contacting the suspension and second aliquot of clear solution with a solvent mixture of ethanol/ethyl acetate and slurried for a first predetermined period of time followed by contacting with ethyl acetate and slurried for a second predetermined period of time. In these embodiments, the first predetermined period of time and the second predetermined period of time may independently be 1 minute or more, such as 2 minutes or more, such as 3 minutes or more, such as 4 minutes or more, such as 5 minutes or more, such as 10 minutes or more, such as 15 minutes or more, such as 30 minutes or more, such as 45 minutes or more, such as 60 minutes or more, such as 2 hours or more, such as 3 hours or more, such as 4 hours or more, such as 8 hours or more, such as 12 hours or more and including 16 hours or more.

In embodiments, the crystalline solid meglumine salts of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid is isolated by filtration (e.g., vacuum filtration) or the solvent may be removed by heating or roto-evaporation. In certain embodiments, the crystalline solid meglumine salts of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid is isolated by filtering, followed by drying at room temperature under nitrogen atmosphere or under vacuum.

The components used in each step of the subject methods may be a purified composition or a crude composition as desired. The term “purified” is used in its conventional sense to refer to a composition where at least some isolation or purification process has been conducted, such as for example, filtration or aqueous workup of a reaction mixture. In certain instances, purification includes liquid chromatography, recrystallization, distillation (e.g., azeotropic distillation) or other type of compound purification. In some embodiments, a mixture is used in a subsequent step in the methods described herein as a crude mixture where no purification or other workup of the reaction mixture has been conducted. In certain instances, the crude composition reaction mixtures include the compound of interest in sufficient purity such as where the crude composition includes a compound of interest in a purity of 90% or greater, such as 95% or greater, such as 97% or greater and including 99% or greater, as determined by high performance liquid chromatography (HPLC), proton nuclear magnetic resonance spectroscopy (¹H NMR) or a combination thereof.

Aspects of the present disclosure include compositions that include one or more of the crystalline solid meglumine salts of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid as described above and a pharmaceutically acceptable excipient. A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) 11. C. Ansel et al., eds 7th ed., Lippincott, Williams, & Wilkins; and Handbook of Pharmaceutical Excipients (2000) A. H. Kibbe et al., eds., 3rd ed. Amer.

Pharmaceutical Assoc. For example, the one or more excipients may include sucrose, starch, mannitol, sorbitol, lactose, glucose, cellulose, tale, calcium phosphate or calcium carbonate, a binder (e.g., cellulose, methylcellulose, hydroxymethylcellulose, polypropylpyrrolidone, polyvinylpyrrolidone, gelatin, gum arabic, poly(ethylene glycol), sucrose or starch), a disintegrator (e.g., starch, carboxymethylcellulose, hydroxypropyl starch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinylpyrrolidone or aluminum stearate), a dispersing agent (e.g., hydroxypropylmethylcellulose), a diluent (e.g., water), and base wax (e.g., cocoa butter, white petrolatum or polyethylene glycol).

The crystalline solid meglumine salts of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid may be formulated into compositions suitable for delivery to a subject by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In certain instances, compositions of interest are formulated for injection such as by subcutaneous injection, intramuscular injection, intravitreal injection, intracisternal injection or intrathecal injection. In other instances, compositions are formulated to be administered orally to the subject. In still other instances, compositions are formulated to be administered intraocularly to the subject. In yet other instances, compositions are formulated to be administered topically or transdermally to the subject.

In some embodiments, compositions of interest include an aqueous buffer. Suitable aqueous buffers include, but are not limited to, acetate, succinate, citrate, and phosphate buffers varying in strengths from about 5 mM to about 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. In some instances, compositions of interest further include a preservative. Suitable preservatives include, but are not limited to, a benzyl alcohol, phenol, chlorobutanol, benzalkonium chloride, and the like. In many cases, the composition is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

In some embodiments, compositions include other additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Where the composition is formulated for injection, the crystalline solid meglumine salts of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid may be formulated by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Although the dosage used in treating a subject will vary depending on the clinical goals to be achieved, a suitable dosage range of the subject compounds is one which provides up to about 0.0001 mg to about 5000 mg, e.g., from about 1 mg to about 25 mg, from about 25 mg to about 50 mg, from about 50 mg to about 100 mg, from about 100 mg to about 200 mg, from about 200 mg to about 250 mg, from about 250 mg to about 500 mg, from about 500 mg to about 1000 mg, or from about 1000 mg to about 5000 mg of an active agent, which can be administered in a single dose. Those of skill will readily appreciate that dose levels can vary as a function of the specific compound, the severity of the symptoms and the susceptibility of the subject to side effects.

In some embodiments, a suitable dose of the crystalline solid meglumine salts of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid is in the range of from about 1 mg/kg body weight to about 500 mg/kg body weight, e.g., from about 5 mg/kg body weight to about 500 mg/kg body weight, from about 10 mg/kg body weight to about 500 mg/kg body weight, from about 20 mg/kg body weight to about 500 mg/kg body weight, from about 30 mg/kg body weight to about 500 mg/kg body weight, from about 40 mg/kg body weight to about 500 mg/kg body weight, from about 50 mg/kg body weight to about 500 mg/kg body weight, from about 60 mg/kg body weight to about 500 mg/kg body weight, from about 70 mg/kg body weight to about 500 mg/kg body weight, from about 80 mg/kg body weight to about 500 mg/kg body weight, from about 90 mg/kg body weight to about 500 mg/kg body weight, from about 100 mg/kg body weight to about 500 mug/kg body weight, from about 200 mug/kg body weight to about 500 mg/kg body weight, from about 300 mg/kg body weight to about 500 mg/kg body weight, or from about 400 mg/kg body weight to about 500 mg/kg body weight.

In some embodiments, a suitable dose of the crystalline solid meglumine salts of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid is in the range of from about 1 mg/kg body weight to about 5 mg/kg body weight, from about 5 mg/kg body weight to about 10 mg/kg body weight, from about 10 mg/kg body weight to about 20 mg/kg body weight, from about 20 mg/kg body weight to about 30 mg/kg body weight, from about 30 mg/kg body weight to about 40 mg/kg body weight, from about 40 mg/kg body weight to about 50 mg/kg body weight, from about 50 mg/kg body weight to about 100 mg/kg body weight, or from about 100 mg/kg body weight to about 500 mg/kg body weight.

In some embodiments, a single dose of a crystalline solid meglumine salt of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid is administered. In other embodiments, multiple doses are administered. Where multiple doses are administered over a period of time, the compound is administered, e.g., twice daily (qid), daily (qd), every other day (qod), every third day, three times per week (tiw), or twice per week (biw) over a period of time. For example, the subject compound is administered qid, qd, qod, tiw, or biw over a period of from one day to about 2 years or more. For example, the compound is administered at any of the aforementioned frequencies for one week, two weeks, one month, two months, six months, one year, or two years, or more, depending on various factors.

Dose units of the present disclosure can be made using manufacturing methods available in the art and can be of a variety of forms suitable for injection (including intracisternal, intrathecal, intravenous, intramuscular, subcutaneous and dermal) administration, for example as a solution, suspension, solution, lyophilate or emulsion. The dose unit can contain components conventional in pharmaceutical preparations, e.g. one or more carriers, binders, lubricants, excipients (e.g., to impart controlled release characteristics), pH modifiers, coloring agents or further active agents.

Dose units provided as liquid dose units can have a total weight of from about 1 microgram to about 1 gram, and can be from about 5 micrograms to 1.5 grams, from about 50 micrograms to 1 gram, from about 100 micrograms to 1 gram, from 50 micrograms to 750 milligrams, and may be from about 1 microgram to 2 grams.

Dose units can comprise components in any relative amounts. For example, dose units can be from about 0.1% to 99% by weight of active ingredients (i.e., crystalline solid meglumine salt compound) per total weight of dose unit. In some embodiments, dose units can be from 10% to 50%, from 20% to 40%, or about 30% by weight of active ingredients per total weight dose unit.

Dose units can be provided in a variety of different forms and optionally provided in a manner suitable for storage. For example, dose units can be disposed within a container suitable for containing a pharmaceutical composition. The container can be, for example, a bottle (e.g., with a closure device, such as a cap, a vial, an ampule (for single dose units), a dropper, thin film, a tube and the like.

Containers can include a cap (e.g., screw cap) that is removably connected to the container over an opening through which the dose units disposed within the container can be accessed.

Containers can include a seal which can serve as a tamper-evident and/or tamper-resistant element, which seal is disrupted upon access to a dose unit disposed within the container. Such seal elements can be, for example, a frangible element that is broken or otherwise modified upon access to a dose unit disposed within the container. Examples of such frangible seal elements include a seal positioned over a container opening such that access to a dose unit within the container requires disruption of the seal (e.g., by peeling and/or piercing the seal). Examples of frangible seal elements include a frangible ring disposed around a container opening and in connection with a cap such that the ring is broken upon opening of the cap to access the dose units in the container.

Liquid dose units can be placed in a container (e.g., bottle or ampule) of a size and configuration adapted to maintain stability of dose units over a period during which the dose units are dispensed into a prescription. For example, containers can be sized and configured to contain 10, 20, 30, 40, 50, 60, 70, 80, 90, 100 or more single liquid dose units. The containers can be sealed or resealable. The containers can packaged in a carton (e.g., for shipment from a manufacturer to a pharmacy or other dispensary). Such cartons can be boxes, tubes, or of other configuration, and may be made of any material (e.g., cardboard, plastic, and the like). The packaging system and/or containers disposed therein can have one or more affixed labels (e.g., to provide information such as lot number, dose unit type, manufacturer, and the like).

The container can include a moisture barrier and/or light barrier, e.g., to facilitate maintenance of stability of the active ingredients in the dose units contained therein. The container can be adapted to contain a single dose unit or multiples of a dose unit. The container can include a dispensing control mechanism, such as a lock out mechanism that facilitates maintenance of dosing regimen.

Dose units can be provided in a container in which they are disposed, and may be provided as part of a packaging system (optionally with instructions for use). For example, dose units containing different amounts of the crystalline solid meglumine salts of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid can be provided in separate containers, which containers can be disposed with in a larger container (e.g., to facilitate protection of dose units for shipment). For example, one or more dose units as described herein can be provided in separate containers, where dose units of different compositions are provided in separate containers, and the separate containers disposed within package for dispensing.

The crystalline solid meglumine salts of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid described herein can be used for prevention or treatment of various ailments, such as a senescence-related condition. Such conditions will typically (although not necessarily) characterized by an overabundance of senescent cells (such as cells expressing p16 and other senescence markers) in or around the site of the condition, or an overabundance of expression of p16 and other senescence markers, in comparison with the frequency of such cells or the level of such expression in unaffected tissue.

In certain embodiments, the crystalline solid meglumine salts of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid described herein can be used for preventing or treating an ophthalmic condition in a subject, whereby at least one sign or symptom of the disease is decreased in severity. Such conditions include both back-of-the-eye diseases, and front-of-the-eye diseases. Diseases of the eye that can be treated according to the present disclosure include presbyopia, macular degeneration (including wet or dry AMD), macular edema, ischemic or vascular conditions such as diabetic retinopathy, glaucomatous retinopathy, ischemic arteritic optic neuropathies, and vascular diseases characterized by arterial and venous occlusion, retinopathy of prematurity and sickle cell retinopathy, glaucoma, degenerative conditions, such as dermatochalasis, ptosis, keratitis sicca, Fuch's corneal dystrophy, presbyopia, cataract, wet age related macular degeneration (wet AMD), dry age related macular degeneration (dry AMD); degenerative vitreous disorders, including vitreomacular traction (VMT) syndrome, macular hole, epiretinal membrane (ERM), retinal tears, retinal detachment, and proliferative vitreoretinopathy (PVR), genetic conditions, such as retinitis pigmentosa, Stargardt disease, Best disease and Leber's hereditary optic neuropathy (LHON), conditions caused by a bacterial, fungal, or virus infection such as conditions caused or provoked by an etiologic agent such as herpes zoster varicella (HZV), herpes simplex, cytomegalovirus (CMV), and human immunodeficiency virus (HIV), inflammatory conditions, such as punctate choroiditis (PIC), multifocal choroiditis (MIC) and serpiginous choroidopathy and iatrogenic conditions, such as a post-vitrectomy cataract and radiation retinopathy.

In other embodiments, the crystalline solid meglumine salts of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid described herein can be developed for treating osteoarthritis in accordance with the present disclosure. Osteoarthritis degenerative joint disease is characterized by fibrillation of the cartilage at sites of high mechanical stress, bone sclerosis, and thickening of the synovium and the joint capsule. Fibrillation is a local surface disorganization involving splitting of the superficial layers of the cartilage. The early splitting is tangential with the cartilage surface, following the axes of the predominant collagen bundles. Collagen within the cartilage becomes disorganized, and proteoglycans are lost from the cartilage surface. In the absence of protective and lubricating effects of proteoglycans in a joint, collagen fibers become susceptible to degradation, and mechanical destruction ensues. Predisposing risk factors for developing osteoarthritis include increasing age, obesity, previous joint injury, overuse of the joint, weak thigh muscles, and genetics. Symptoms of osteoarthritis include sore or stiff joints, particularly the hips, knees, and lower back, after inactivity or overuse; stiffness after resting that goes away after movement; and pain that is worse after activity or toward the end of the day.

In still other embodiments, the crystalline solid meglumine salts of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid described herein can be used to reduce or inhibit loss or erosion of proteoglycan layers in a joint, reduces inflammation in the affected joint, and promotes, stimulates, enhances, or induces production of collagen, for example, type 2 collagen. The compound may cause a reduction in the amount, or level, of inflammatory cytokines, such as IL-6, produced in a joint and inflammation is reduced. The compounds can be used for treating osteoarthritis and/or inducing collagen, for example, Type 2 collagen, production in the joint of a subject. A compound also can be used for decreasing, inhibiting, or reducing production of metalloproteinase 13 (MMP-13), which degrades collagen in a joint, and for restoring proteoglycan layer or inhibiting loss and/or degradation of the proteoglycan layer. Treatment with a compound thereby may also reduce the likelihood of, inhibits, or decreases erosion, or slows erosion of the bone. The compound may be administered directly to an osteoarthritic joint, for example, intra-articularly, topically, transdermally, intradermally, or subcutaneously. The compound may also restore, improve, or inhibit deterioration of strength of a join, and reduce joint pain.

In still other embodiments, the crystalline solid meglumine salts of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid described herein be used for preventing or treating a pulmonary disease in a subject. Pulmonary conditions that can be treated according to the present disclosure include idiopathic pulmonary fibrosis (IPF), chronic obstructive pulmonary disease (COPD), asthma, cystic fibrosis, bronchiectasis, and emphysema.

In certain embodiments, the crystalline solid meglumine salts of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid described herein can be used to treat senescence-related conditions, such as those described in International Patent Publication No. WO 2019/213160, the disclosure of which is herein incorporated by reference.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. By “average” is meant the arithmetic mean. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1—Salts of (R)-5-(4-chlorophenyl)-1-isopropyl-2-methyl-4-(3-(4-(4-(4-((1-(phenylthio)-4-(4-((phosphonooxy)methyl)piperidin-1-yl)butan-2-yl)amino)-3-((trifiuoromethyl)sulfonyl)phenyl)sulfonamidolphenyl)piperazin-1-yl)phenyl-1-H-pyrrole-3-carboxylic acid

Different salts were prepared from the free acid of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid. Free acid compounds were purified and were amorphous. Forming the salt compounds was tested in 8 different bases (KOH, NaOH, meglumine, L-arginine, ammonia, nicotinamide, L-lysine and calcium acetate). Low-crystallinity or amorphous salts are obtained in L-arginine, ammonia, nicotinamide, L-lysine and calcium acetate. The sodium and potassium salts of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid were unstable. The meglumine salt showed high crystallinity and high solubility in water.

Materials and Methods

Appropriate amount of 8 bases were dissolved and diluted to 10 mL with different solvent combinations (e.g., MeOH or MeOH/Water) to make 0.1 M solution. (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid was dissolved with MeOH or THF/W to make 20 or 30 mg/mL solution. Compound solutions were distributed into 96-well plates. Each well contained 100 μL of free acid solution and 26 μL or 72 μL, of each base solution. After evaporated to dryness, 200 μL of solvent was added. Wells were covered with a parafilm with one pinhole and evaporated under ambient conditions. One sample of each line was characterized by 1H NMR to confirm the formation of salts. Solid samples obtained were characterized by XRPD to find out whether they are crystalline. The bases and solvents used are shown in Tables 1 and 2 below.

TABLE 1
Bases

| | |
|---|---|
| Potassium hydroxide (KOH) | Sodium hydroxide (NaOH) |
| L-arginine | Ammonia |
| Nicotinamide | L-lysine |
| Meglumine | Calcium acetate |

TABLE 2
Solvents

| | |
|---|---|
| Methanol (MeOH) | Ethanol (EtOH) |
| Isopropanol (IPA) | Isobutanol |
| Water (W) | Acetonitrile (ACN) |
| Acetone | 2-Butanone |
| Isopropyl acetate (IPAc) | Ethyl Acetate (EA/EtOAc) |
| Methyl tert-butyl ether (MTBE) | Tetrahydrofuran (THF) |
| Dichloromethane (DCM) | |

Analysis Methods

X-ray Powder Diffraction (XRPD)—Solid samples were examined using D8 ADVANCE X-ray diffractometer (Bruker). The diffractometer was equipped with LynxEye detector. In XRPD analysis, samples were scanned from 3 to 40° 2θ at a step of 0.02° 2θ. The tube voltage and current were 40 KV and 40 mA, respectively.

Polarized Light Microscope (PLM)—PLM analysis was conducted with a polarized light microscope ECLIPSE LV100POL (Nikon, JPN).

Thermogravimetric Analysis (TGA)—TGA was carried out on Discovery TGA 55 (TA Instruments, US). The sample was placed in an open tarred aluminum pan, automatically weighed, and inserted into the TGA furnace. The sample was heated at 10° C./min to the final temperature.

Differential Scanning Calorimeter (DSC) DSC analysis was conducted with Discovery DSC 250 (TA Instruments, US). A weighted sample was placed into a DSC pinhole pan, and the weight was accurately recorded. The sample was heated at 10° C./min to the final temperature.

Dynamic Vapor Sorption (DVS)—DVS was determined using IGA Sorp (Hiden Isochema, UK). The sample was tested at a targeted RH of 0 to 90% full cycle in step mode. The analysis was performed in 10% RH increments.

Results

In a 96-well plate, 1 eq. or 3 eq. of 0.1 M base along with free acid solution was added into a well, respectively. After drying, some solid samples appeared in 96-well plate. One sample in each row of 96-well plate was analyzed by 1H-NMR, and solid samples were characterized by PLM and XRPD.

Chemical shift was observed by NMR for all samples, indicating successful formation of salts. No crystallinity was observed for the samples with 1 eq. of base. The samples of sodium and calcium salt with 3 eq. base are crystalline with low crystallinity.

Calcium salt shows similar diffraction peaks to calcium acetate on XRPD pattern, suggesting that calcium salt might not be prepared. The rest of samples are all amorphous.

Preparation of Salts

Preparation of potassium salt—Potassium salt was prepared with either 1 eq. or 3 eq. of KOH. The results are summarized in Table 3 below. XRPD results show that form 1 and form 2 were prepared in THF/W/EtOH and MeOH/W/IPA respectively. After slurry in acetone or heptane, the samples became amorphous. Form 1 shows about 4.7% weight loss prior to 190° C. by TGA. Two endothermic peaks at 138.43° C. and 217.52° C. were observed by DSC, suggesting form 1 might be a solvate. Form 2 was only obtained in a small amount and was not further characterized. Free acid was not stable with presence of strong bases.

TABLE 3
Preparation of Potassium Salt

| No. | Ratio | Solvent Vₛ/Vₐₛ | Result |
|---|---|---|---|
| 1 | 1 eq. | (THF/W)/MeOH 3/1 | PLM: No birefringence |
| 2 | 3 eq. | (THF/W)/EtOH 5/1 | XRPD: Nearly amorphous |
| 3 | 3 eq. | (THF/W)/MeOH 10/1 | XRPD: Nearly amorphous |
| 4 | 3 eq. | (THF/W)/EtOH | XRPD: Form 1; TGA: 4.7%/ |
| | | | 190° C. |
| 5 | 3 eq. | (MeOH/W)/IPA 1/2 | DSC: T_{endo}: 138.43° C. and |
| | | | 217.52° C. |
| 6 | 3 eq. | (MeOH/W)/IPA 1/2 | PLM: No birefringence |
| 7 | 3 eq. | (MeOH/W)/MeOH 3/1 | XRPD: Nearly amorphous |

Preparation of arginine salt—Arginine salt was prepared with either 1 eq. or 3 eq. of L-arginine. The results are summarized in Table 4 below. No arginine salts were able to be prepared.

TABLE 4
Preparation of Arginine Salt

| No. | Ratio | Solvent Vₛ/Vₐₛ | Result |
|---|---|---|---|
| 1 | 1 eq. | (THF/W)/EtOH | PLM: microcrystalline |
| 2 | 1 eq. | (THF/W)/EtOH 3/1 | PLM: No birefringence |
| 3 | 3 eq. | (THF/W)/EtOH | PLM: No birefringence |
| | | | XRPD: Amorphous |

Preparation of sodium salt—Sodium salt was prepared with either 1 eq. or 3 eq. of NaOH. The results are summarized in Table 5 below. Crystalline solid was prepared from THF/W/EtOH and named as Form 1. Mono-sodium salt was originally prepared by process chemistry with a high purity of >99.0%. The salt and free acid were not stable in the presence of strong base.

TABLE 5
Preparation of Sodium Salt

| No. | Ratio | Solvent Vₛ/Vₐₛ | Result |
|---|---|---|---|
| 1 | 1 eq. | (THF/W)/MeOH 3/1 | PLM: No birefringence |
| 2 | 3 eq. | (THF/W)/EtOH 5/1 | PLM: No birefringence |
| 3 | 3 eq. | (THF/W)/EtOH | PLM: Irregular crystalline |
| | | | XRPD: Form 1 |

Preparation of meglumine salt—Meglumine salt was prepared with either 1 eq. or 3 eq. of meglumine. The results are summarized in Table 6 below. XRPD results showed that the form prepared in MeOH/THF/W/EtOH/EA was repeatable and named as Form 1. Form 1 shows about 0.9% weight loss prior to 130° C. by TGA. Two endothermic peaks at 84.1 and 147.4° C. were observed by DSC, suggesting form 1 might be an anhydrate with little solvent residue. DVS analysis of form 1 shows water sorption of 16.1% from 0% to 80% RH (23% at 90% RH).

TABLE 6
Preparation of Meglumine Salt

| No. | Ratio | Solvent Vₛ/Vₐₛ | Result |
|---|---|---|---|
| 1 | 1 eq. | (THF/W)/MeOH 3/1 | PLM: No birefringence |
| 2 | 3 eq. | EtOH/EA 2/3 | PLM: No birefringence |
| 3 | 3 eq. | MeOH/THF/W/EtOH/EA | XRPD: Form 1; TGA: 0.9%/ |
| | | 9/9/1/5/8 | 130° C. |
| | | | DSC: T_{endo}: 84.1° C. and |
| | | | 147.4° C. |
| | | | DVS: 0-23% absorption between |
| | | | 0% to 90% RH. Crystallinity |
| | | | decreased after DVS test. |

Preparation of calcium salt—Calcium salt was prepared with either 1 eq. or 3 eq. of calcium acetate. The results are summarized in Table 7 below. Crystalline solid was obtained. XRPD shows that the characteristic diffraction peaks of calcium salt are similar to calcium acetate, suggesting calcium salt may be not prepared by reaction.

TABLE 7
Preparation of Calcium Salt

| No. | Ratio | Solvent Vₛ/Vₐₛ | Result |
|---|---|---|---|
| 1 | 1 eq. | (THF/W)/EtOH | PLM: Irregular crystal and |
| | | | no birefringence |
| 2 | 1 eq. | (THF/W)/EtOH 3/1 | PLM: Weak birefringence |
| 3 | 3 eq. | (THF/W)/EtOH 1/1 | PLM: Weakly birefringence |
| | | | XRPD: Form 1 |

Preparation of ammonium salt—Ammonium salt was prepared with either 1 eq. or 3 eq. of ammonium. The results are summarized in Table 8 below. No solid of ammonium salt was obtained.

TABLE 8
Preparation of Ammonium Salt

| No. | Ratio | Solvent Vₛ/Vₐₛ | Result |
|---|---|---|---|
| 1 | 1 eq. | (THF/W)/EtOH 5/8 | Oil |
| 2 | 3 eq. | (THF/W)/EtOH 5/8 | Oil |

Preparation of nicotinamide salt—Nicotinamide salt was prepared with either 1 eq. or 3 eq. of nicotinamide. The results are summarized in Table 9 below. No solid of nicotinamide salt was obtained.

TABLE 9
Preparation of Nicotinamide Salt

| No. | Ratio | Solvent Vₛ/Vₐₛ | Result |
|---|---|---|---|
| 1 | 1 eq. | (THF/W)/EtOH 5/8 | Oil |
| 2 | 3 eq. | (THF/W)/EtOH 5/8 | Oil |

Preparation of lysine salt—Lysine salt was prepared with either 1 eq. or 3 eq. of lysine. The results are summarized in Table 10 below. No solid of lysine salt was obtained.

TABLE 10
Preparation of Lysine Salt

| No. | Ratio | Solvent Vₛ/Vₐₛ | Result |
|---|---|---|---|
| 1 | 1 eq. | (THF/W)/EtOH 5/8 | Oil |
| 2 | 3 eq. | (THF/W)/EtOH 5/8 | Oil |
| 2 | 3 eq. | (THE/W)/EtOH | Oil |

### CONCLUSIONS

Salts were prepared from the free acid of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid with 8 bases, including KOH, NaOH, L-arginine, meglumine, calcium acetate, ammonium, nicotinamide and L-lysine. Three crystalline salts (potassium salt, sodium salt and meglumine salt) were obtained with sodium salt and meglumine salt showing good crystallinity. However, free acid was not stable in the presence of KOH or NaOH. Meglumine salt is chemically stable under experimental conditions and shows good crystallinity and high solubility in water (˜26 mg/mL).

### Example 2—Polymorphs of crystalline solid meglumine salts of (R)-5-(4-chlorophenyl)-1-isopropyl-2-methyl-4-(3-(4-(4-(4-((1-(phenylthio)-4-(4-((phosphonooxy)methyl)piperidin-1-yl)butan-2-yl)amino)-3-((trifluoromethyl)sulfonyl)phenyllsulfonamido)phenyl)piperazin-1-yl)phenyl)-1H-pyrrole-3-carboxylic acid

Polymorphs of crystalline solid meglumine salts of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifliuoromethyl)sulfonyl)phenyl)sulfonamido)phenyl)piperazin-1-yl)phenyl)-1H-pyrrole-3-carboxylic acid were prepared. Six different forms exhibited crystallinity and were characterized by XRPD, TGA, DSC, DVS and HPLC. A summary of the properties of the six isolated forms is listed in Table 11.

TABLE 11
Polymorphs

| | | | TGA, | | |
|---|---|---|---|---|---|
| | | | wt. | | |
| | Base:Acid | | prior to | | HPLC (%) |
| Forms | (NMR) | DSC | 130° C. | DVS | 60° C., 0/7 d |
| I-Solvate or | N/A | 68/84° C.; 18/J/g | 1% | 15.3% (0- | N/A |
| hydrate | | 139/147° C.; 11 J/g | | 80% RH) | |
| | | | | 23% (0-90% RH) | |
| II-Solvate | 1:2.7 | 104/110° C.; 1.3 | 2% | N/A | 97.74/97.02 |
| or hydrate | | J/g | | | |
| | | 126/136° C.; 23 J/g | | | |
| III-Hydrate | 1:2.7 | 108/113° C.; 1 J/g | 2% | 9.4% (0-80% RH) | 98.09/97.7 |
| | | 131/142° C.; 33 J/g | | 22% (0-90% RH) | |
| IV- | 1:3 | 135/143° C.; 44 J/g | 0% | 9.1% (0-80% RH) | 98.76/98.7 |
| Anhydrate | | | | 15.4% (0- | |
| | | | | 90% RH) | |
| V-Hydrate | 1:3 | 95/115° C.; 14 J/g | 1.2% | 6.8% (0-80% RH) | 97.72/97.31 |
| | | 136/143° C.; 35 J/g | | 14.5% (0- | |
| | | | | 90% RH) | |
| VI-Solvate | 1:2.7 | 92/110° C.; 22 J/g | 1% | 8.2% (0-80% RH) | 98.32/97.95 |
| or hydrate | | 133/142° C.; 46 J/g | | 18.2% (0- | |
| | | | | 90% RH) | |

Materials and Methods

Solvents for screening—Solvents for preparing and screening the properties of the polymorphs are listed in Table 12.

TABLE 12
Solvents for Screening

| | |
|---|---|
| Methanol (MeOH) | Ethanol (EtOH) |
| Isopropanol (IPA) | Tert butyl alcohol (TBA) |
| Water (W) | Acetonitrile (ACN) |
| Acetone | Butanone |
| Isopropyl acetate (IPAc) | Ethyl Acetate (EA/EtOAc) |
| Methyl tert-butyl ether (MTBE) | Tetrahydrofuran (THF) |
| Dichloromethane (DCM) | Methyl isobutyl ketone (MIBK) |

Solubility estimation—Preliminary solubility studies of each meglumine salt compound was carried out by visual observation. A list of solvents tested is shown in Table 13.

TABLE 13
Solvents for Estimating Solubility

| No. | Solvent |
|---|---|
| 1 | Acetonitrile (ACN) |
| 2 | Methyl tert-butyl ether (MTBE) Tert butyl alcohol |
| | (TBA) |
| 3 | Ethyl Acetate (EA/EtOAc) |
| 4 | Isopropanol (IPA) Butanone |
| 5 | Water |
| 6 | Tetrahydrofuran (THF) |
| 7 | Dichloromethane (DCM) Ethanol (EtOH) |
| 8 | Acetone Tert butyl alcohol (TBA) |
| 9 | Isopropyl acetate (IPAc) |
| 10 | Methyl isobutyl ketone (MIBK) |
| 11 | Methanol (MeOH) |
| 12 | Ethanol (EtOH) |
| 13 | 2-Butanone |

Reaction crystallization—Meglumine salt was prepared by using different ratios of free acid and meglumine. Crystalline material was attempted to obtain by using different solvents.

Crystallization by slurry—Appropriate amount of sample was added into solvent to make a suspension. The suspension was kept stirring or shaking at room temperature or higher temperature. Solid sample was collected for XRPD analysis after certain intervals.

Cooling crystallization—Appropriate amount of sample was added into solvent to make suspension which was kept stirring at room temperature or higher temperature. Solid sample was collected for XRPD analysis after certain intervals.

Competitive slurry—The mixture of two or more crystal forms was suspended in specific solvent at fixed temperature. If the solubility was high in the solvent or the amount of one form is very small, the solvent will be saturated with other crystal forms at first to make sure no crystal form will be completely dissolved before saturation. The suspension will be sampled after certain time interval to check the polymorphic conversion.

Solid Stability Test—Appropriate amount of meglumine salt was placed at 60° C. and 40° C./75% RH for up to one week, and sampled at 0, 3 and 7 days. The sample was dissolved in diluent to prepare solution at 0.5 mg/mL for HPLC analysis. Solid samples were analyzed by XRPD to check the crystal form.

Analysis Methods

X-ray Powder Diffraction (XRPD)—Solid samples were examined using D8 ADVANCE X-ray diffractometer (Bruker). The diffractometer was equipped with LynxEye detector. In XRPD analysis, samples were scanned from 3 to 40° 20 at a step of 0.02° 2θ. The tube voltage and current were 40 KV and 40 mA, respectively.

Polarized Light Microscope (PLM)—PLM analysis was conducted with a polarized light microscope ECLIPSE LV100 POL (Nikon, JPN).

Thermogravimetric Analysis (TGA)—TGA was carried out on Discovery TGA 55 (TA Instruments, US). The sample was placed in an open tarred aluminum pan, automatically weighed, and inserted into the TGA furnace. The sample was heated at 10° C./min to the final temperature.

Differential Scanning Calorimeter (DSC)—DSC analysis was conducted with Discovery DSC 250 (TA Instruments, US). A weighted sample was placed into a DSC pinhole pan, and the weight was accurately recorded. The sample was heated at 10° C./min to the final temperature.

Dynamic Vapor Sorption (DVS)—DVS was determined using IGA Sorp (Hiden Isochema, UK). The sample was tested at a targeted RH of 0 to 90% full cycle in step mode. The analysis was performed in 10% RH increments.

HPLC—High performance liquid chromatography was performed as summarized in Table 14

TABLE 14
HPLC

| Instrument | Agilent 1260 Series |
|---|---|
| Column | Xselect CSH Fluoro-Phenyl, 150 *4.6 mm, 3.5 μm |
| Injection Volume | 5 μL |
| Wavelength | 257 nm |
| Injection Conc. | 1 mg/mL |
| Mobile Phase | A: 0.7% H3PO4 in H2O; B: 0.7% H3PO4 in ACN |
| T/B % | 0/30, 1.0/30, 28.0/60, 29.0/90, 32.0/90, 33.0/30, 40.0/30 |
| Temperature | 30° C. |
| Diluent | MeOH:H2O = 9:1 |

Results

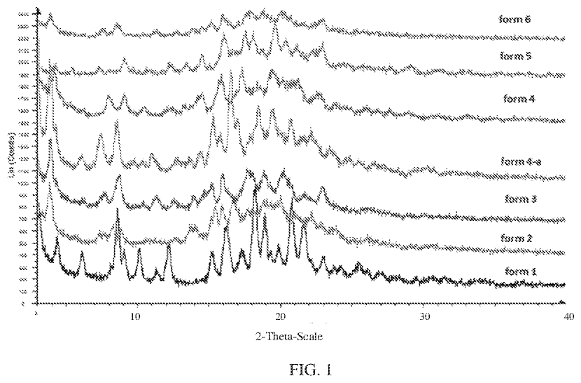

Summary of Prepared Crystal Forms—7 forms were identified and defined as Forms I, II, III, IVA, IV, V and VI. The XRPD patterns of all the discovered forms are presented in FIG. 1 and the preparation methods are shown in Table 15. Form I was prepared with low purity free acid (95.8%) and repeated with purer material (99.1%). Among the prepared crystalline solid meglumine salts of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid forms, Forms II, III, IVA, V and VI were identified as either hydrate or solvate. Form IV was identified as being an anhydrate.

TABLE 15
Summary of Prepared Crystal Forms

| Form | Definition | Agilent 1260 Series |
|---|---|---|
| I | Solvate/ | Reaction crystallization in MeOH/THF/W/ |
| | hydrate | EtOH/EA |
| II | Solvate/ | Reaction crystallization in MeOH/THF/W/ |
| | hydrate | EtOH/EA |
| III | Hydrate | Slurry crystallization in IPAC/W/EtOH system |
| IVA | Solvate/ | Reaction crystallization inTHE/W/EtOH/EA |
| | hydrate | |
| IV | Anhydrate | Reaction crystallization inTHE/W/EtOH/EA |
| V | Hydrate | Slurry crystallization in ACN-W system |
| VI | Hydrate/ | Slurry crystallization in Acetone-W system |
| | Solvate | |

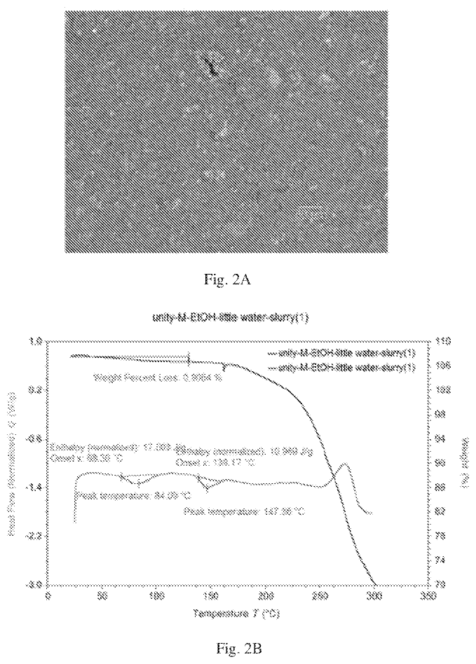

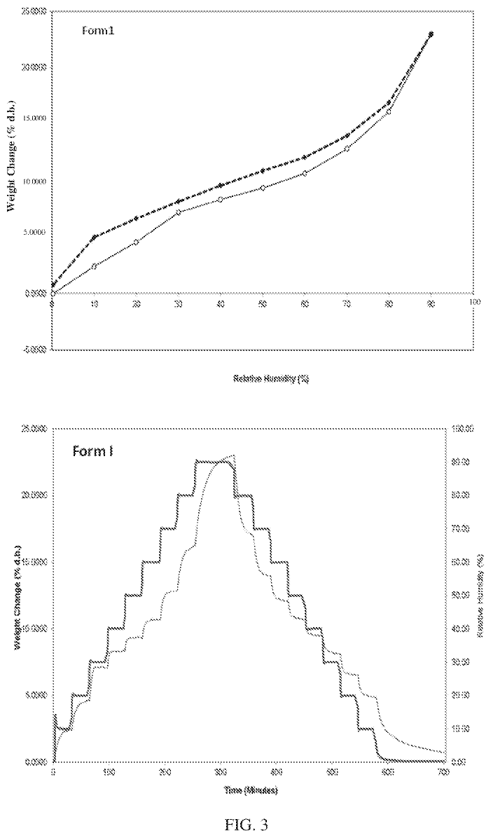

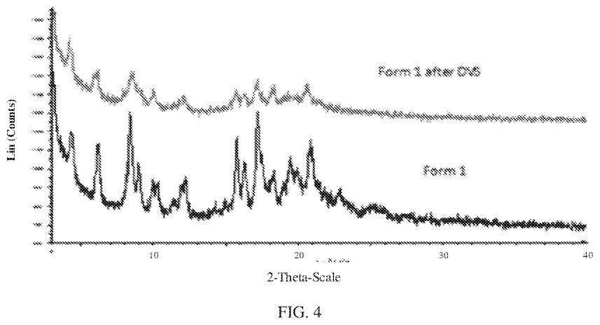

Characterization of Form I—Form I shows irregular crystals with good crystallinity by PLM (FIG. 2A) and XRPD (FIG. 4). TGA in FIG. 2B shows that there is ˜0.9% weight loss between RT to 130° C. Two endothermic peaks at 84° C. and 147° C. were observed by DSC which may be due to solvent evaporation and melting respectively. DVS analysis (FIG. 3) shows that form I absorbed ˜15.3% moisture from 0% to 80% RH (23%, 0-90% RH), so form I is very hygroscopic. Crystallinity decreased after DVS test as shown by the XRPD depicted in FIG. 4. Table 16 lists the 20 peaks of the XRPD of Form L.

TABLE 16
XRPD 2-Theta Peaks of Form I

| 2-Theta (°) | Intensity (%) | Intensity (Count) | d Value (Angstrom) |
|---|---|---|---|
| 4.349 | 58.5 | 553 | 20.30002 |
| 6.102 | 45.4 | 429 | 14.47202 |
| 8.101 | 34.3 | 324 | 10.90511 |
| 8.585 | 83.6 | 790 | 10.29122 |
| 9.042 | 47.1 | 445 | 9.77206 |
| 10.1 | 47.2 | 446 | 8.75065 |
| 11.301 | 30.4 | 287 | 7.82377 |
| 12.154 | 50.3 | 475 | 7.27613 |
| 15.218 | 44.2 | 418 | 5.81754 |
| 16.245 | 69.7 | 659 | 5.45183 |
| 17.283 | 46 | 435 | 5.12667 |
| 18.21 | 100 | 945 | 4.86783 |
| 18.905 | 79.2 | 748 | 4.69035 |
| 19.313 | 45.3 | 428 | 4.59222 |
| 19.881 | 51.5 | 487 | 4.46219 |
| 20.772 | 88.4 | 835 | 4.27287 |
| 21.618 | 72.3 | 683 | 4.1074 |
| 22.105 | 37.8 | 357 | 4.01817 |
| 23.035 | 40.2 | 380 | 3.85784 |
| 23.78 | 31.2 | 295 | 3.73876 |
| 24.231 | 32.1 | 303 | 3.6702 |
| 25.197 | 29.6 | 280 | 3.53162 |
| 25.505 | 33.1 | 313 | 3.48959 |
| 26.085 | 28.6 | 270 | 3.41334 |
| 27.109 | 27.4 | 259 | 3.28671 |
| 29.512 | 23 | 217 | 3.02433 |
| 32.646 | 18.7 | 177 | 2.74076 |

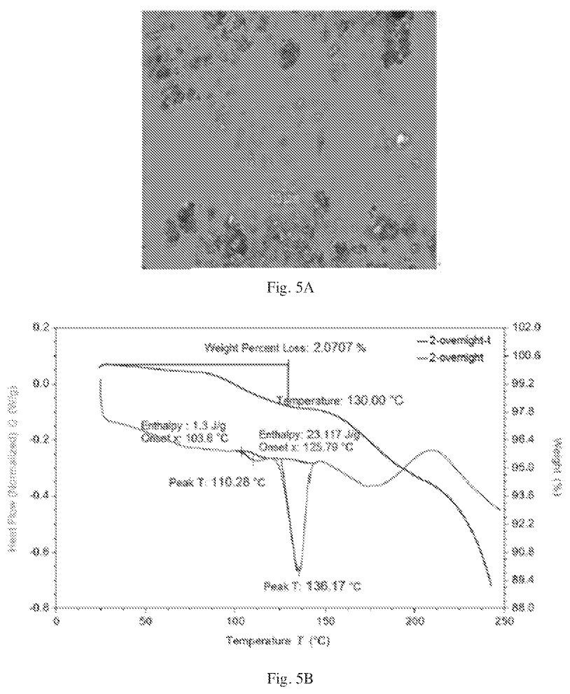

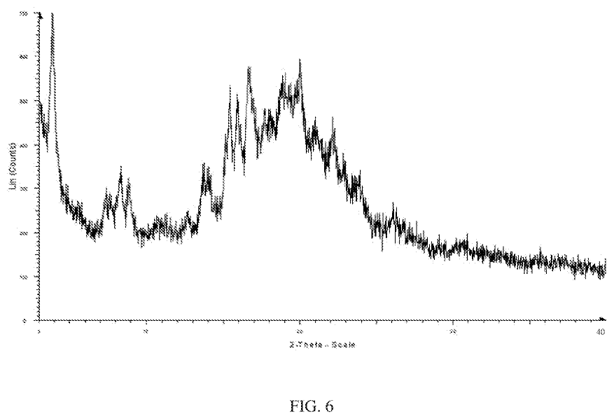

Characterization of Form II—Form II shows irregular morphology with low crystallinity by PLM (FIG. 5A) and XRPD (FIG. 6). TGA in FIG. 5B shows a weight loss of 2% between RT to 130° C. An endothermic peak at 136° C. was observed by DSC which is likely due to melting of Form II. The ratio of free acid to meglumine was calculated as 1 to 2.7 according to 1H-NMR. Table 17 lists the 2θ peaks of the XRPD of Form II.

TABLE 17
XRPD 2-Theta Peaks of Form II

| 2-Theta (°) | Intensity (%) | Intensity (Count) | d Value (Angstrom) |
|---|---|---|---|
| 3.825 | 100 | 701 | 23.07993 |
| 7.319 | 42.2 | 296 | 12.06916 |
| 8.257 | 50.1 | 351 | 10.69896 |
| 8.831 | 44.8 | 314 | 10.00553 |
| 13.715 | 47.8 | 335 | 6.45141 |
| 15.152 | 60.5 | 424 | 5.84261 |
| 15.44 | 76 | 533 | 5.73442 |
| 15.928 | 73.6 | 516 | 5.55969 |
| 16.657 | 82.6 | 579 | 5.31809 |
| 17.703 | 68.8 | 482 | 5.00592 |
| 18.84 | 75.3 | 528 | 4.70647 |
| 19.997 | 84.9 | 595 | 4.43661 |
| 22.149 | 66 | 463 | 4.01028 |
| 23.862 | 46.8 | 328 | 3.726 |

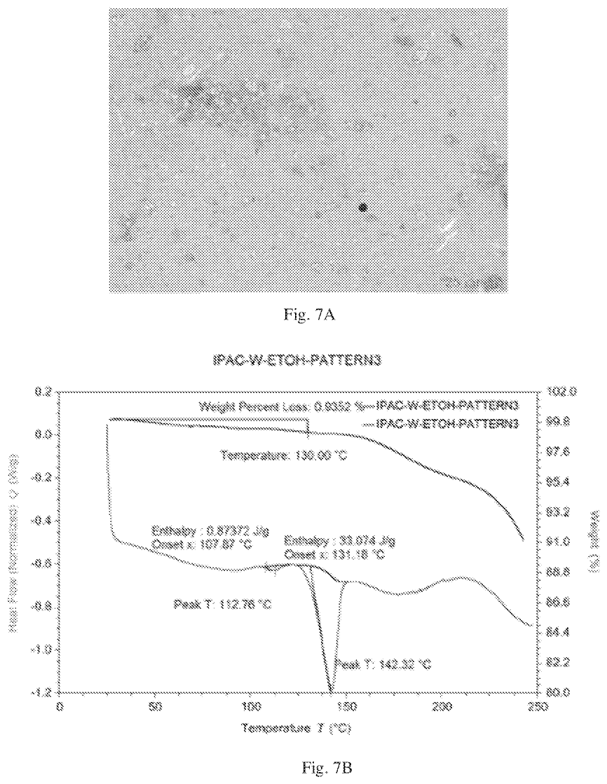

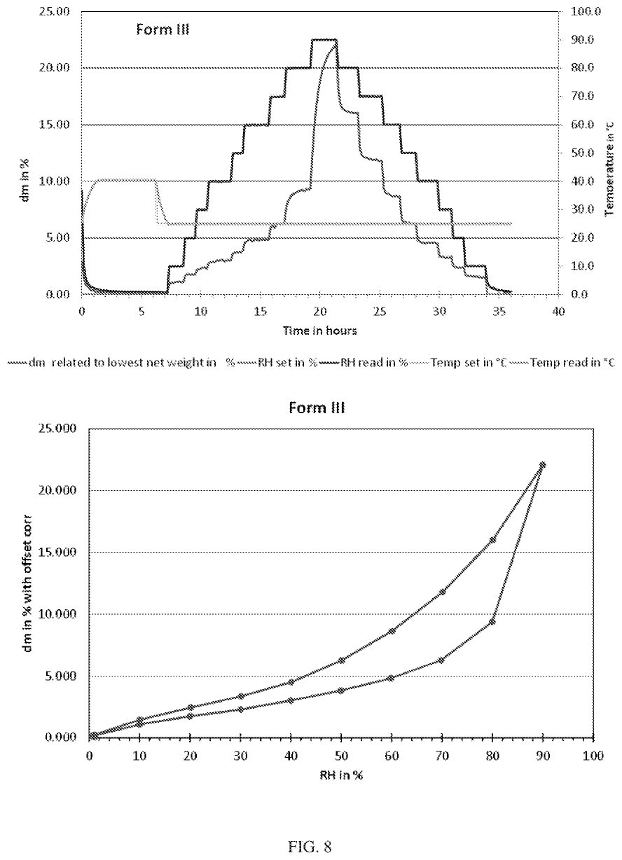

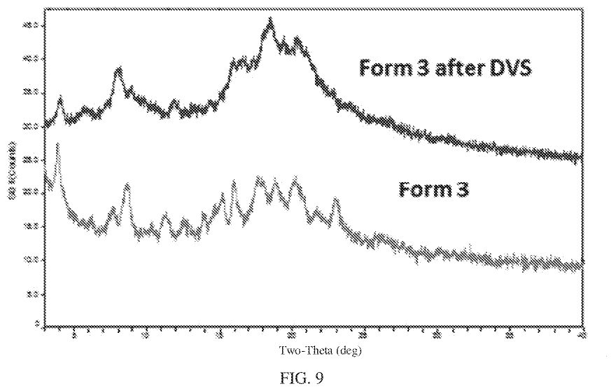

Characterization of Form III—Form III shows irregular crystals with low crystallinity (FIG. 7A). TGA in FIG. 7B shows 0.9% weight loss between RT to 130° C. DSC profile displays a small endothermic event at 113° C. followed by a big endothermic peak at 142° C. Form IV (described below) was obtained by heating Form III to 130° C. Chemical shift of meglumine CI-13 in 1H-NMR spectrum was observed, indicating salt formation. The ratio of free acid to meglumine was calculated as 1 to 2.7. DVS (FIG. 8) shows that form III absorbs ˜9.4% moisture from 0% to 80% RH (˜22%, 0-90% RH). Form III is hygroscopic. Crystallinity decreased after DVS by the XRPD depicted in FIG. 9. Table 18 lists the 20 peaks of the XRPD of Form III.

TABLE 18
XRPD 2-Theta Peaks of Form III

| 2-Theta (°) | Intensity (%) | Intensity (Count) | d Value (Angstrom) |
|---|---|---|---|
| 3.872 | 100 | 762 | 22.8035 |
| 4.263 | 54.1 | 412 | 20.70849 |
| 6.096 | 35.8 | 273 | 14.48602 |
| 7.451 | 38.3 | 292 | 11.85481 |
| 7.696 | 42.4 | 323 | 11.47835 |
| 8.72 | 60.5 | 461 | 10.13228 |
| 10.366 | 32.5 | 248 | 8.52722 |
| 11.308 | 36.2 | 276 | 7.81859 |
| 11.489 | 34.8 | 265 | 7.69607 |
| 11.548 | 32.5 | 248 | 7.65682 |
| 12.518 | 33.7 | 257 | 7.06555 |
| 13.943 | 37.9 | 289 | 6.34652 |
| 14.697 | 44.5 | 339 | 6.02231 |
| 15.195 | 52.4 | 399 | 5.82638 |
| 15.947 | 64.2 | 489 | 5.55313 |
| 17.691 | 67.2 | 512 | 5.00928 |
| 18.04 | 63.6 | 485 | 4.91319 |
| 18.833 | 63.9 | 487 | 4.70816 |
| 20.179 | 61.4 | 468 | 4.39698 |
| 21.716 | 42 | 320 | 4.08911 |
| 22.959 | 49.2 | 375 | 3.8706 |
| 25.795 | 23.9 | 182 | 3.451 |

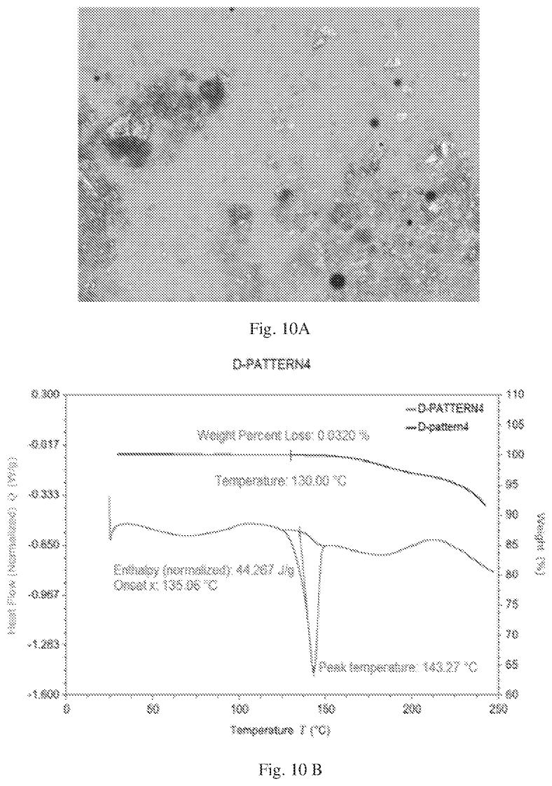

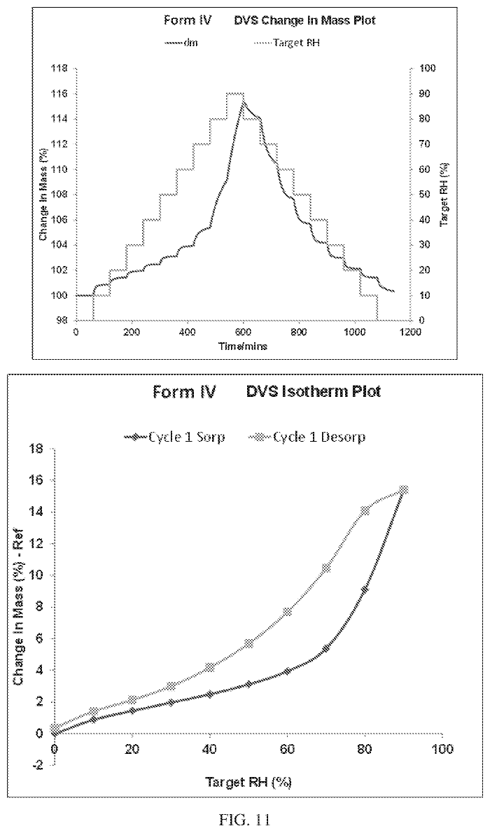

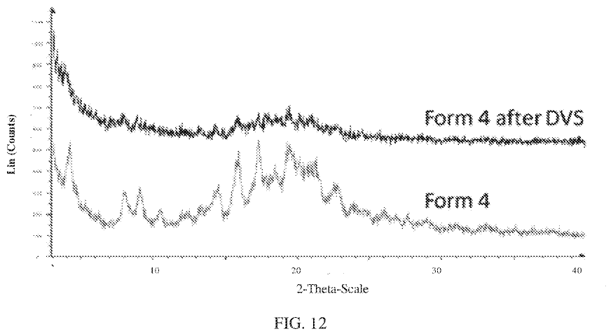

Characterization of Form IV—Form IV shows irregular crystals with high crystallinity by PLM (FIG. 10A) and XRPD (FIG. 12). TGA in FIG. 10B did not show significant weight loss prior to 130° C. DSC profile shows an endothermic peak at 143.3° C. which is due to melting of Form IV. Significant chemical shift of meglumine CH₃ confirms salt formation, and 1:3 ratio free acid to meglumine was calculated. DVS result in FIG. 11 shows that Form IV absorbs 9.1% moisture from 0% to 80% RH (˜15.4%, 0-90% RH). Form IV is hygroscopic. Crystallinity decreased after DVS by the XRPD depicted in FIG. 12. Table 19 lists the 20 peaks of the XRPD of Form IV.

TABLE 19
XRPD 2-Theta Peaks of Form IV

| 2-Theta (°) | Intensity (%) | Intensity (Count) | d Value (Angstrom) |
|---|---|---|---|
| 4.16 | 98.4 | 539 | 21.22539 |
| 4.635 | 54.4 | 298 | 19.05055 |
| 7.936 | 56.8 | 311 | 11.1314 |
| 9.073 | 59.7 | 327 | 9.73937 |
| 10.445 | 40.9 | 224 | 8.46262 |
| 13.303 | 43.4 | 238 | 6.65042 |
| 14.489 | 61.5 | 337 | 6.10863 |
| 15.798 | 82.3 | 451 | 5.60505 |
| 16.767 | 58.8 | 322 | 5.28331 |
| 17.292 | 100 | 548 | 5.12407 |
| 19.474 | 96.5 | 529 | 4.5547 |
| 19.603 | 91.4 | 501 | 4.52486 |
| 20.15 | 83.9 | 460 | 4.4034 |
| 27.685 | 37.2 | 204 | 3.21958 |

Characterization of Form IVA—Table 20 lists the 20 peaks of the XRPD of Form IVA.

TABLE 20
XRPD 2-Theta Peaks of Form IVA

| 2-Theta (°) | Intensity ( %) | Intensity (Count) | d Value (Angstrom) |
|---|---|---|---|
| 3.836 | 100 | 1128 | 23.01433 |
| 4.221 | 52.6 | 593 | 20.91624 |
| 6.073 | 30.3 | 342 | 14.54151 |
| 7.383 | 44.2 | 499 | 11.96475 |
| 8.554 | 54 | 609 | 10.32932 |
| 10.334 | 25.1 | 283 | 8.55364 |
| 10.94 | 30.7 | 346 | 8.08097 |
| 12.701 | 25.8 | 291 | 6.96386 |
| 13.69 | 26.9 | 303 | 6.46326 |
| 14.424 | 31.9 | 360 | 6.13598 |
| 15.295 | 59.6 | 672 | 5.78842 |
| 15.745 | 47.3 | 533 | 5.62376 |
| 16.517 | 92.1 | 1039 | 5.36276 |
| 17.013 | 55.9 | 630 | 5.20761 |
| 17.867 | 43.8 | 494 | 4.96039 |
| 18.451 | 66.1 | 746 | 4.80482 |
| 19.467 | 64.5 | 727 | 4.55614 |
| 20.719 | 56.7 | 640 | 4.28372 |
| 22.162 | 46.7 | 527 | 4.0078 |
| 22.459 | 39.6 | 447 | 3.95553 |
| 23.426 | 35.1 | 396 | 3.79444 |
| 24.838 | 27.2 | 307 | 3.58178 |
| 28.234 | 18.9 | 213 | 3.15824 |

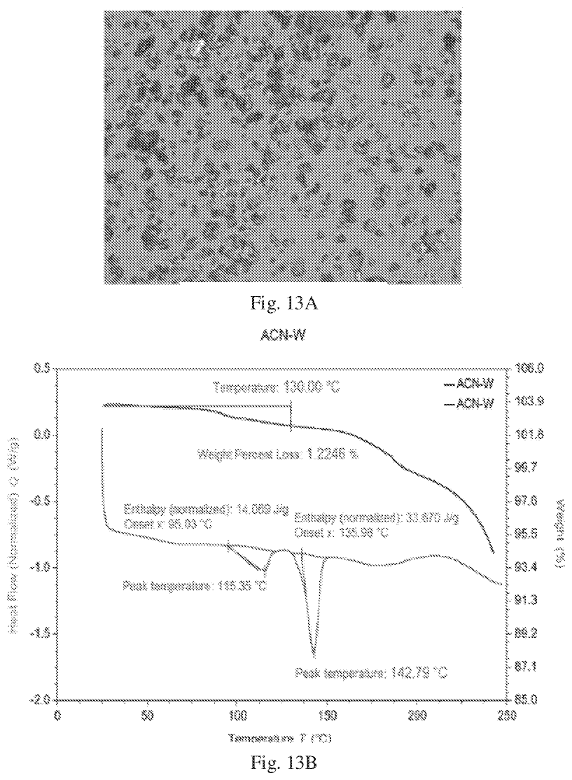

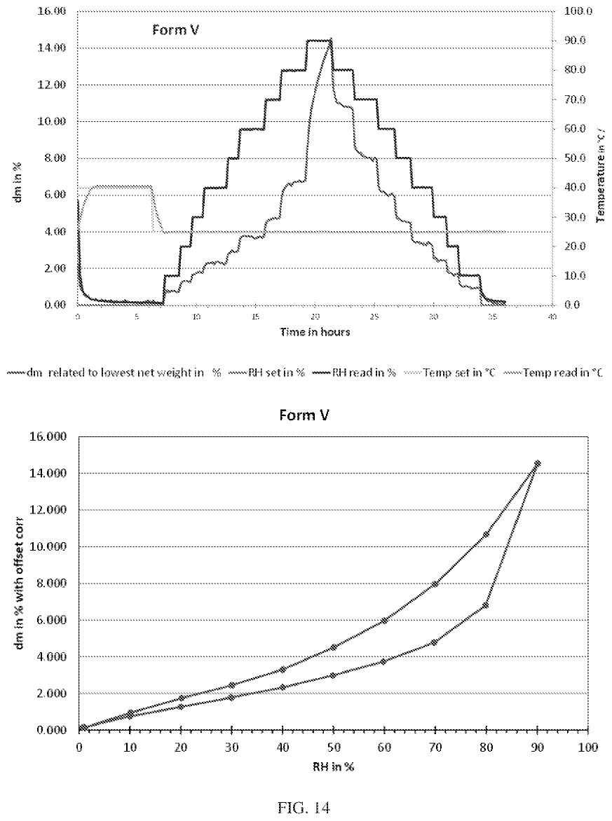

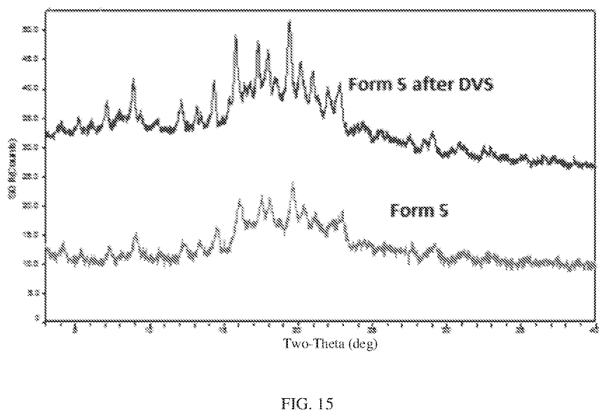

Characterization of Form V—Form V shows irregular crystals with high crystallinity by PLM (FIG. 13A) and XRPD (FIG. 15). TGA in FIG. 13B shows that there is 1.2% weight loss between RT to 130° C. DSC result shows two endothermic peaks at 115° C. and 143° C. No residual solvent was detected by NMR for Form V, and Form IV was obtained by heating form V to 130° C. Meglumine CH₃ exhibits a chemical shift in 1H-NMR, indicating salt formation. The ratio of free acid to meglumine was calculated as 1:3. DVS (FIG. 14) shows that Form V absorbed ˜6.8% moisture from 0% to 80% RH (˜14.5%, 0-90% RH). Form V is hygroscopic. The crystal form remains unchanged and the crystallinity slightly increased after DVS shown by the XRPD depicted in FIG. 15. Table 21 lists the 20 peaks of the XRPD of Form V.

TABLE 21
XRPD 2-Theta Peaks of Form V

| 2-Theta (°) | Intensity (%) | Intensity (Count) | d Value (Angstrom) |
|---|---|---|---|
| 4.189 | 29.9 | 173 | 21.07696 |
| 5.4 | 27.5 | 159 | 16.3532 |
| 7.305 | 30.1 | 174 | 12.09127 |
| 9.068 | 41.8 | 242 | 9.74404 |
| 12.187 | 35.6 | 206 | 7.2566 |
| 12.374 | 32.5 | 188 | 7.14747 |
| 13.369 | 34.7 | 201 | 6.61764 |
| 14.509 | 46.1 | 267 | 6.10026 |
| 16.053 | 76 | 440 | 5.51653 |
| 17.541 | 81.9 | 474 | 5.05204 |
| 18.099 | 76.9 | 445 | 4.8975 |
| 18.759 | 54.6 | 316 | 4.72658 |
| 19.633 | 100 | 579 | 4.51801 |
| 20.365 | 65.6 | 380 | 4.35731 |
| 21.152 | 61.3 | 355 | 4.19695 |
| 22.282 | 53.7 | 311 | 3.98654 |
| 22.96 | 65.3 | 378 | 3.87041 |
| 27.643 | 32.8 | 190 | 3.2244 |
| 29.183 | 32.3 | 187 | 3.05768 |

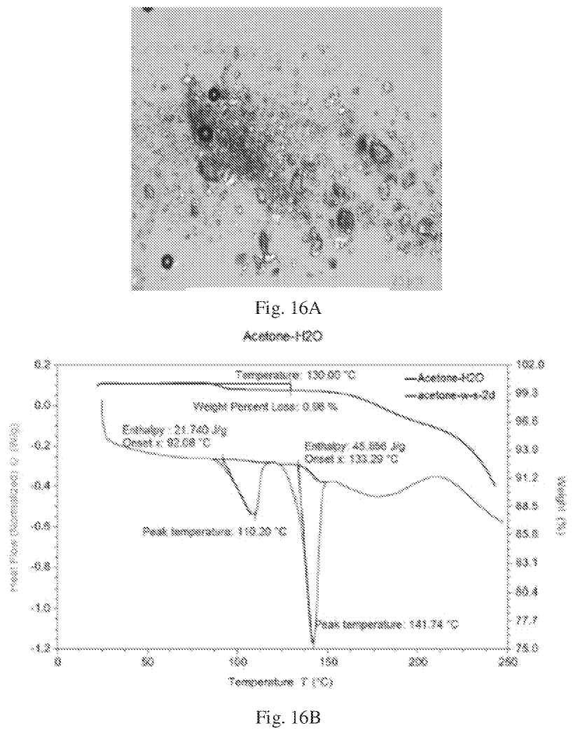

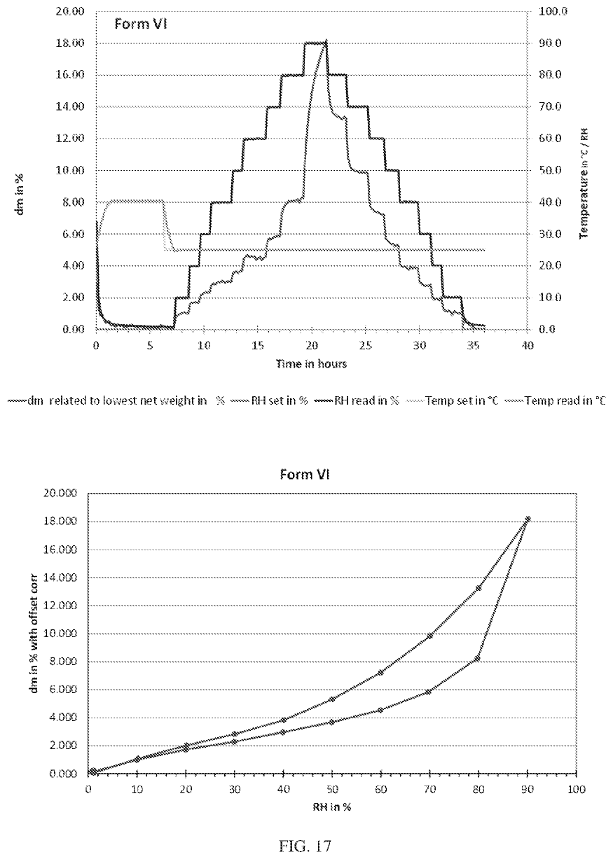

Characterization of Form VI—Form VI shows irregular crystals with low crystallinity by PLM (FIG. 16A) and XRPD (FIG. 18). TGA in FIG. 16B shows that there is 1% weight loss prior to 130° C. DSC profile shows two endothermic peaks at 110 and 142° C. Form VI converted to Form IV after being heated to 130° C. Meglumine CH₃ exhibits a chemical shift indicating salt formation. A ratio of 1:2.7 free acid to meglumine was calculated according to NMR. DVS in FIG. 17 shows that Form VI absorbed ˜8.2% moisture from 0% to 80% RH (˜18.2%, 0-90% RH). Form VI is hygroscopic. Crystallinity decreased after DVS by the XRPD depicted in FIG. 18. Table 22 lists the 2θ peaks of the XRPD of Form VI.

TABLE 22
XRPD 2-Theta Peaks of Form VI

| 2-Theta (°) | Intensity (%) | Intensity (Count) | d Value (Angstrom) |
|---|---|---|---|
| 3.862 | 93.9 | 278 | 22.85848 |
| 8.476 | 65.2 | 193 | 10.42374 |
| 8.613 | 61.1 | 181 | 10.25788 |
| 8.692 | 59.1 | 175 | 10.16508 |
| 11.292 | 45.9 | 136 | 7.82986 |
| 12.719 | 43.6 | 129 | 6.95452 |
| 13.86 | 48.6 | 144 | 6.38427 |
| 14.499 | 52 | 154 | 6.10409 |
| 15.137 | 65.5 | 194 | 5.84827 |
| 15.947 | 89.5 | 265 | 5.55311 |
| 17.608 | 97 | 287 | 5.03288 |
| 17.693 | 92.2 | 273 | 5.00891 |
| 18.814 | 100 | 296 | 4.71296 |
| 20.046 | 99.3 | 294 | 4.42587 |
| 20.694 | 85.8 | 254 | 4.28872 |
| 22.993 | 79.1 | 234 | 3.86494 |
| 35.07 | 30.4 | 90 | 2.5567 |
| 36.116 | 28.4 | 84 | 2.48503 |
| 36.751 | 26.7 | 79 | 2.44351 |

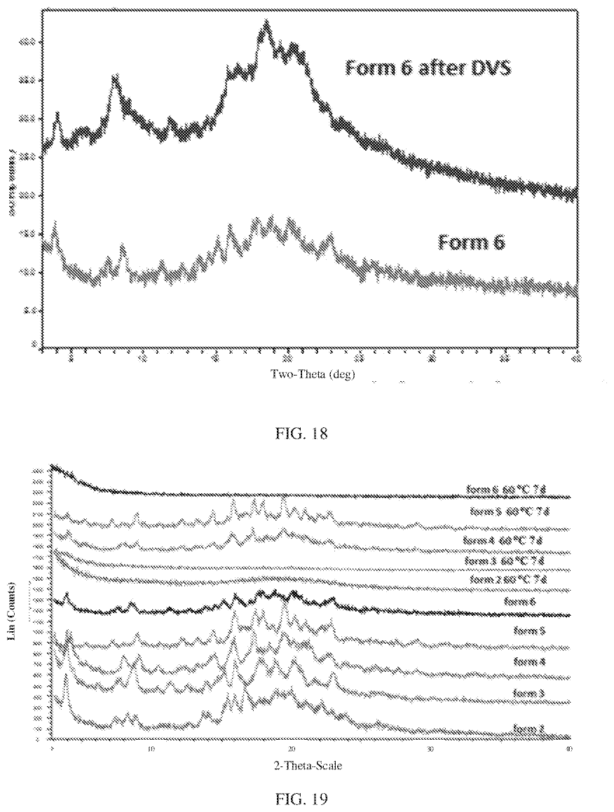

Solid-State Stability—The solid-state stability study of form II, III, IV, V and VI was carried out at 60° C. for up to 7 days. The sample was analyzed by XRPD (FIG. 19) and HPLC (Table 23) at 0 and 7 days. Form IV shows the highest purity among all the forms and was found as the most stable form at 60° C. for 7 days. The other forms show minor degradation after stored at 60° C. for one week. Amorphous was obtained for Forms II, III and VI after one-week storage at 60° C. The crystal form of Forms IV and V remain unchanged.

TABLE 23
Stability Test measured by HPLC

| | HPLC purity/% | | |
|---|---|---|---|
| Form | 60° C.-Day 0 | 60° C.-Day 3 | 60° C.-Day 7 |
| II | 97.74 | 97.67 | 97.02 |
| III | 98.09 | 97.78 | 97.70 |
| IV | 98.76 | 98.63 | 98.70 |
| V | 97.72 | 97.35 | 97.31 |
| VI | 98.32 | 98.07 | 97.95 |

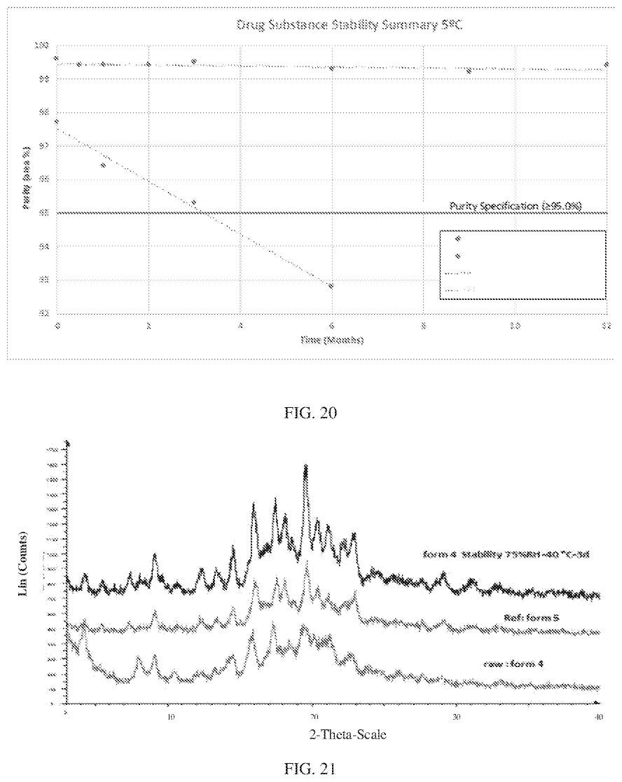

The stability of a crystalline solid meglumine salt of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid over 12 months was tested as compared to a sodium salt of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazirn-1-yl)phenyl)-1H-pyrrole-3-carboxylic acid. As shown in FIG. 20, the meglumine salt compound exhibits little to no change in purity throughout the 12 month test period. On the other hand, the sodium salt exhibits a sharp decrease in purity and falls below 95% purity within 3 months. The 12 month stability of the Form IV of the meglumine salt of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid was also studied by HPLC (HPLC conditions summarized in Table 24). As shown in Table 25, the Form IV of the meglumine salt shows little change through 12 months.

TABLE 24
HPLC Conditions for 12-month Stability Test

| Instrument | Agilent 1260 Series |
|---|---|
| Column | Xselect CSH Fluoro-Phenyl, 150 *4.6 mm, 3.5 μm |
| Injection Volume | 5 μL |
| Wavelength | 257 nm |
| Injection Conc. | 1 mg/mL |
| Mobile Phase | A: 0.7% H₃PO₄ in H₂O; B: 0.7% H₃PO₄ in ACN |
| T/B % | 0/30, 1.0/30, 28.0/60, 29.0/90, 32.0/90, 33.0/30, 40.0/30 |
| Temperature | 30° C. |
| Diluent | MeOH:H₂O = 9:1 |

TABLE 25
HPLC Results for 12-month Stability Test
Meglumine Salt Stablity (2-8° C.)

| | | 2 | 1 | 2 | 3 | 6 | 9 | 12 |
|---|---|---|---|---|---|---|---|---|
| RRT | T₀ | weeks | month | months | months | months | months | months |
| 0.86 | 0.06 | <0.05 | 0.05 | 0.05 | 0.09 | 0.05 | 0.08 | 0.08 |
| 0.94 | 0.07 | 0.07 | 0.07 | 0.07 | <0.05 | 0.06 | 0.06 | 0.06 |
| 1 | 99.6 | 99.4 | 99.4 | 99.4 | 99.5 | 99.3 | 99.2 | 99.4 |
| 1.05 | <0.05 | <0.05 | <0.05 | <0.05 | <0.05 | 0.05 | 0.07 | <0.05 |
| 1.09 | 0.06 | 0.07 | 0.07 | 0.08 | 0.08 | 0.06 | 0.09 | <0.05 |
| 1.15 | <0.05 | <0.05 | <0.05 | <0.05 | <0.05 | <0.05 | <0.05 | 0.07 |
| 1.27 | <0.05 | <0.05 | <0.05 | <0.05 | <0.05 | <0.05 | 0.1 | 0.09 |
| 1.29 | 0.22 | 0.38 | 0.38 | 0.40 | 0.36 | 0.44 | 0.41 | 0.14 |
| 1.65 | <0.05 | <0.05 | ND | ND | <0.05 | <0.05 | <0.05 | 0.18 |

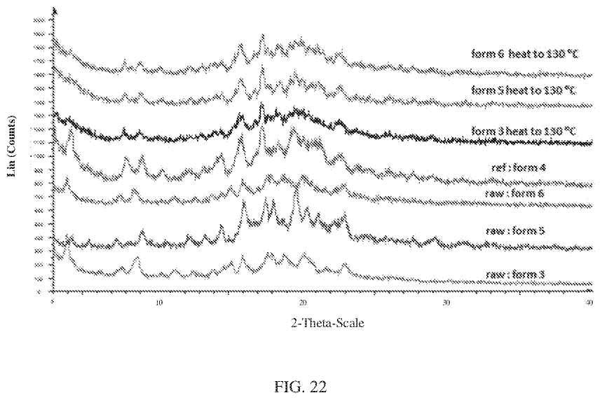

The solid-state stability of Form IV at 40° C./75% RH was also studied. The sample was analyzed by XRPD after 3 days. The XRPD result in FIG. 21 shows that Form IV can be converted from Form V. A thermal treatment study was carried out for Forms III, V and VI. Samples were heated to 130° C. with a ramping rate of 5° C./min, and then analyzed by XRPD (FIG. 22). Forms III, V and VI converted to Form IV upon heating.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A crystalline solid meglumine salt of a compound of Formula I:

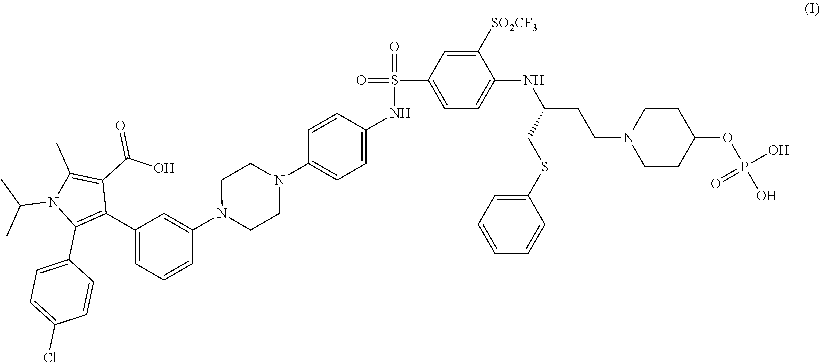

2. The crystalline solid according to claim 1, wherein meglurnine is present in the crystalline solid in a stoichiornetric ratio of from 1 to 3.

3. The crystalline solid according to claim 1, wherein the crystalline solid is stable at a temperature of from 2° C. to 8° C. for 12 months or more.

4.-27. (canceled)

28. Form IV of a crystalline solid meglumine salt of a compound of Formula I:

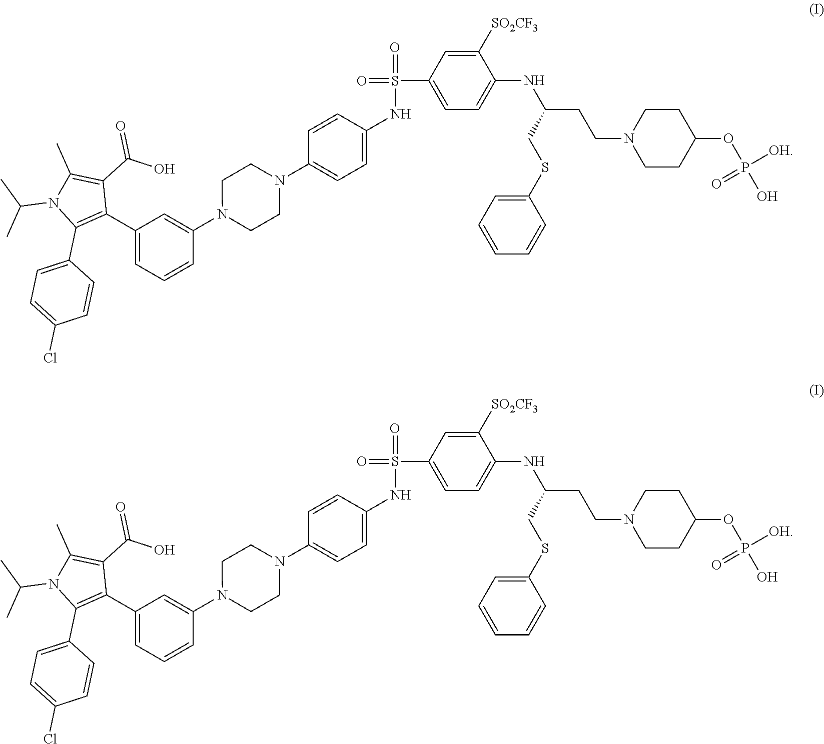

29. The crystalline solid according to claim 28, wherein meglumine is present in the crystalline solid in a stoichiometric ratio of from 1 to 3.

30. The crystalline solid according to claim 28, having an x-ray powder diffraction pattern comprising one or more peaks at about 4.20° 2θ; about 4.60° 2θ; about 7.90° 2θ; about 9.1° 2θ; about 10.4° 2θ; about 13.3° 2θ; about 14.5° 2θ; about 15.8° 2θ; about 16.8° 2θ; about 17.3° 2θ; about 19.5° 2θ; about 19.6° 2θ; about 20.2° 2θ; or about 27.7° 2θ.

31. The crystalline solid according to claim 28, wherein Form IV of the crystalline solid meglumine salt of a compound of Formula I is characterized by a single weight loss step at about 130° C. by thermograVimetric analysis (TGA).

32. The crystalline solid according to claim 28, wherein Form IV of the crystalline solid meglumine salt of a compound of Formula I exhibits a first endotherm at about 130° C. and a second endotherm at about 143.3° C. by differential scanning calorimetry (DSC).

33. The crystalline solid according to claim 28, wherein the crystalline solid is stable at a temperature of from 2° C. to 8° C. for 12 months or more.

34-45. (canceled)

46. A method of making a crystalline solid meglumine salt compound of claim 1, the method comprising:
generating a clear solution comprising a meglumine salt of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3 -((trifluoromethyl)sulfonyl)phenyl)sulfonamido)phenyl)piperazin-1-yl)phenyl)-1H-pyrrole-3-carboxylic acid;
contacting an aliquot of the clear solution with a seed composition and a solvent composition to generate a first suspension;
contacting the first suspension with a second aliquot of the clear solution and a solvent composition to generate a slurry composition; and
filtering a crystalline solid meglumine salt of (R)-5-(4-chlorophenyl)-1-isopropyl-2-methyl-4-(3-(4-(4-((4-((1-(phenylthio)-4-(4- ((phosphonooxy)methyl)piperidin-1-yl)butan-2-yl)amino)-3-((trifluoromethyl) sulfonyl)phenyl)sulfonamido)phenyl)piperazin-1-yl)phenyl)-1H-pyrrole-3 -carboxylic acid from the slurry composition.

47. The method according to claim 46, wherein the method comprises: contacting meglumine and (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid in a first solvent composition to generate a first solution comprising solubilized (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl) sulfonyl)phenyl)sulfonamido)phenyl)piperazin-1-yl)phenyl)-1H-pyrrole-3-carboxylic acid meglumine salt;
contacting the first composition with a second solvent composition to generate a clear solution;
contacting a first aliquot of the clear solution with a third solvent composition and a seed composition to generate a first suspension;
contacting the first suspension with a fourth solvent composition to generate a second suspension;
contacting the second suspension with a fifth solvent composition to generate a third suspension;
contacting a second aliquot of the clear solution and a sixth solvent composition with the third suspension to generate a slurry precursor composition;
contacting the slurry precursor composition with a seventh solvent composition to generate a slurry composition; and
filtering a crystalline solid meglumine salt of (R)-5-(4-chlorophenyl)-1-isopropyl-2-methyl-4-(3-(4-(4-((4-((1-(phenylthio)-4-(4-((phosphonooxy)methyl)piperidin-1-yl)butan-2-yl)amino)-3-((trifluoromethyl)sulfonyl)phenyl)sulfonamido)phenyl)piperazin-1-yl)phenyl)-1H-pyrrole-3-carboxylic acid from the slurry composition.

48.-72. (canceled)

73. A composition comprising:
a crystalline solid meglumine salt according to claim 1; and
a pharmaceutically acceptable excipient.

74.-94. (canceled)
